(19) United States
(12) Patent Application Publication
(10) Pub. No.: US 2019/0175052 A1
(43) Pub. Date: Jun. 13, 2019

(54) METHOD FOR RECORDING DIAGNOSTIC MEASUREMENT DATA OF A HEART OF AN EXAMINATION OBJECT IN A HEART IMAGING BY MEANS OF A MAGNETIC RESONANCE DEVICE

(71) Applicant: Siemens Healthcare GmbH, Erlangen (DE)

(72) Inventors: Christoph FORMAN, Erlangen (DE); Michaela SCHMIDT, Uttenreuth (DE)

(73) Assignee: Siemens Healthcare GmbH, Erlangen (DE)

(21) Appl. No.: 16/323,557

(22) PCT Filed: Aug. 4, 2017
(86) PCT No.: PCT/EP2017/069825

§ 371 Date: Feb. 6, 2019

(30) Foreign Application Priority Data
Aug. 12, 2016 (DE) 10 2016 215 112.6

(51) Int. Cl. A61B 5/055; G01R 33/56; A61B 5/00; G01R 33/54

(52) U.S. Cl. CPC A61B 5/055; G01R 33/5601; G01R 33/56325; A61B 5/742; G01R 33/543; A61B 5/0044

(57) ABSTRACT

A method is for recording diagnostic measurement data of a heart of an examination object in a heart imaging via a magnetic resonance device. A magnetic resonance device and a non-transitory computer readable medium are also disclosed. An embodiment of the method for recording diagnostic measurement data of a heart of an examination object in a heart imaging via a magnetic resonance device includes: carrying out of at least two overview recordings of the heart of the examination object, wherein overview measurement data is acquired, and carrying out of at least two diagnostic recordings of the heart of the examination object based on the acquired overview measurement data, wherein diagnostic measurement data is acquired in the at least two diagnostic recordings.

### PRIORITY STATEMENT

This application is the national phase under 35 U.S.C. § 371 of PCT International Application No. PCT/EP2017/069825 which has an International filing date of Aug. 4, 2017, which designated the United States of America and which claims priority to German Patent Application No. DE 102016215112.6 filed Aug. 12, 2016, the entire contents of which are hereby incorporated herein by reference.

### FIELD

Embodiments of the invention relates to a method for recording diagnostic measurement data of a heart of an examination object in a heart imaging via a magnetic resonance device, to a magnetic resonance device and to a computer program product.

### BACKGROUND

In a magnetic resonance device, also referred to as a magnetic resonance tomography system, the body of an object to be examined, for example of a patient, of a healthy test subject, of an animal or of a phantom, is usually exposed with the aid of a basic magnet to a relatively high basic magnetic field, for example of 1.5 or 3 or 7 Tesla. In addition gradient circuits are applied with the aid of a gradient coil unit. High-frequency radio-frequency pulses, for example excitation pulses, are then sent out via suitable antenna devices via a radio-frequency antenna unit, which leads to the nuclear spin of specific atoms resonantly excited by this radio-frequency field being flipped by a defined flip angle in relation to the magnetic field lines of the basic magnetic field. During the relaxation of the nuclear spin radio-frequency signals, so-called magnetic resonance signals, are emitted, which are received via suitable radio-frequency antennas and are then further processed. Finally the desired image data can be reconstructed from the raw data thus acquired.

### SUMMARY

Magnetic resonance imaging can be used to particular advantage in heart imaging in order to record diagnostic image data of a heart of the examination object. At least one embodiment of the invention specifies an improved method for heart imaging via a magnetic resonance device. Advantageous embodiments are described in the claims.

At least one embodiment of the inventive method for recording diagnostic measurement data of a heart of an examination object in a heart imaging via a magnetic resonance device comprises:

carrying out a number of overview recordings of the heart of the examination object, wherein overview measurement data is acquired in the number of overview recordings; and

carrying out a number of diagnostic recordings of the heart of the examination object based on the acquired overview measurement data, wherein diagnostic measurement data is acquired in the number of diagnostic recordings.

At least one embodiment of the inventive magnetic resonance device comprises a measurement data acquisition unit and a processing unit, wherein the magnetic resonance device is designed to carry out at least one embodiment of an inventive method.

Thus the processing unit in particular is embodied to carry out computer-readable instructions, in order to execute at least one embodiment of the inventive method. In particular the magnetic resonance device comprises a memory unit, wherein computer-readable information is stored in the memory unit, wherein the processing unit is embodied to load the computer-readable information from the memory unit and to execute the computer-readable information, in order to carry out at least one embodiment of an inventive method.

In accordance with one form of embodiment, the magnetic resonance device, in particular the measurement data acquisition unit and the processing unit, is embodied so that the heart imaging is a first heart imaging and the number of diagnostic recordings exclusively comprise the following diagnostic recordings:

A first diagnostic recording, which is embodied as a dynamic heart recording along long axis measurement slices of the heart, and

A second diagnostic recording, which is embodied as a dynamic heart recording along short axis measurement slices of the heart.

At least one embodiment of the inventive computer program product is able to be loaded directly into a memory of a programmable processing unit of a magnetic resonance device and has program code segments/modules for carrying out at least one embodiment of an inventive method, when the computer program product is executed in the processing unit of the magnetic resonance device. The computer program product can be a computer program or can include a computer program. This enables at least one embodiment of the inventive method to be carried out quickly, in an identically repeatable manner and robustly.

### BRIEF DESCRIPTION OF THE DRAWINGS

The invention will be described and explained in greater detail below on the basis of the example embodiments shown in the figures, in which:

FIG. 1 shows an execution sequence of a first heart imaging,

FIG. 2 shows an execution sequence of a second heart imaging,

FIG. 3 shows an execution sequence of a third heart imaging,

FIG. 4 shows a magnetic resonance device for carrying out the heart imagings and

FIG. 5 show a selection system, which makes it possible for a user to select a heart imaging to be carried out.

General preparatory remarks relating to the description of the heart imagings

### DETAILED DESCRIPTION OF THE EXAMPLE EMBODIMENTS

At least one embodiment of the inventive method for recording diagnostic measurement data of a heart of an examination object in a heart imaging via a magnetic resonance device comprises:

carrying out a number of overview recordings of the heart of the examination object, wherein overview measurement data is acquired in the number of overview recordings; and

carrying out a number of diagnostic recordings of the heart of the examination object based on the acquired overview measurement data, wherein diagnostic measurement data is acquired in the number of diagnostic recordings.

One form of embodiment makes provision for the at least two overview recordings and the at least two diagnostic recordings to be carried out at least partly nested in one another in their temporal execution sequence.

One form of embodiment makes provision, in the heart imaging, before the temporally first diagnostic recording of the number of diagnostic recordings, for there to be more than twice as many overview recordings as there are overview recordings between the temporally first diagnostic recording of the number of diagnostic recordings and the temporally second diagnostic recording of the number of diagnostic recordings.

One form of embodiment makes provision for the number of overview recordings to amount to a maximum of six.

One form of embodiment makes provision for the temporally first diagnostic recording of the number of diagnostic recordings and the temporally second diagnostic recording of the number of diagnostic recordings to be carried out along different heart axes of the examination object.

One form of embodiment makes provision for measurement slices orthogonal to one another in the heart of the examination object to be acquired in the temporally first diagnostic recording of the number of diagnostic recordings and for measurement slices in parallel to one another in the heart of the examination object to be acquired in the temporally second diagnostic recording of the number of diagnostic recordings.

One form of embodiment makes provision for planning of the measurement slices in parallel to one another to be based on the measurement slices orthogonal to one another acquired in the temporally first diagnostic recording.

One form of embodiment makes provision for there to be a number of measurement blocks with overview recordings before the beginning of a measurement block with the temporally first diagnostic recording of the number of diagnostic recordings, wherein the number of measurement blocks with the overview recordings, totaled up, last more than twice as long as the measurement block with the temporally first diagnostic recording.

One form of embodiment makes provision, at the beginning of the heart imaging, for there to be at least one overview measurement for positioning the heart in an isocenter of the magnetic resonance device and at least one overview measurement for defining an orientation and/or a recording region of long axis measurement slices.

One form of embodiment makes provision for the at least one measurement block with the at least one overview measurement for defining the orientation and/or the recording region of long axis measurement slices to last for a longer time than the at least one measurement block with the at least one overview measurement for positioning the heart in the isocenter of the magnetic resonance device.

One form of embodiment makes provision for the carrying out of at least a part of the number of diagnostic recordings to comprise use of a compressed sensing acceleration technique.

One form of embodiment makes provision for there to be a maximum of five user interactions during the heart imaging.

One form of embodiment makes provision for a combined figure for the number of overview recordings and the number of diagnostic recordings to be twice as large as a figure for the number of user actions that occur during the heart imaging.

One form of embodiment makes provision for there to be precisely one user interaction between the temporally first diagnostic recording of the number of diagnostic recordings and the temporally second diagnostic recording of the number of diagnostic recordings.

One form of embodiment makes provision for there to be at least twice as many user interactions before the beginning of the temporally first diagnostic recording of the number of diagnostic recordings as there are user interactions between the temporally first diagnostic recording and the temporally second diagnostic recording of the number of diagnostic recordings.

One form of embodiment makes provision for there to be more automatic evaluation steps than there are user interactions during the heart imaging.

One form of embodiment makes provision for suggestions to be automatically presented to a user for a user interaction needed, which will simply be accepted or modified by the user for the user interaction.

One form of embodiment makes provision for instructions for the user interaction and/or suitable tools for the user interaction to be provided automatically to the user on a display unit for a user interaction needed.

One form of embodiment makes provision for a maximum imaging duration for the heart imaging to be predetermined, wherein imaging parameters for the heart imaging are only able to be set by a user such that the maximum imaging duration will not be exceeded with the set imaging parameters.

One form of embodiment makes provision for the heart imaging to be a first heart imaging and for the number of diagnostic recordings exclusively to comprise the following diagnostic recordings:

A first diagnostic recording, which is embodied as a dynamic heart recording along long axis measurement slices of the heart, and

A second diagnostic recording, which is embodied as a dynamic heart recording along short axis measurement slices of the heart.

One form of embodiment makes provision for a first maximum imaging duration, which amounts to a maximum of 12 minutes, to be predetermined for the first heart imaging.

One form of embodiment makes provision for the first maximum imaging duration to amount to a maximum of 6 minutes.

One form of embodiment makes provision for the second diagnostic recording to follow on from the first diagnostic recording in time in the first heart imaging.

One form of embodiment makes provision, in the first heart imaging, for the short axis measurement slices to be planned based on the diagnostic measurement data acquired in the first diagnostic recording.

One form of embodiment makes provision, in the first heart imaging, for more than twice as many short axis measurement slices to be acquired in the second diagnostic recording as there are long axis measurement slices acquired in the first diagnostic recording.

One form of embodiment makes provision, in the first heart imaging, for a figure for the number of overview recordings to be at least twice as large as a figure for the number of diagnostic recordings.

One form of embodiment makes provision for the first heart imaging to be carried out without application of contrast medium.

One form of embodiment makes provision, in the first heart imaging, for the measurement block with the second diagnostic recording to have a shorter duration than the measurement block with the first diagnostic recording.

One form of embodiment makes provision, in the first heart imaging, for the measurement blocks with the overview recordings, totaled up, to have a longer duration than is needed by the measurement blocks with the diagnostic recordings.

One form of embodiment makes provision for the start of the measurement block with the first diagnostic recording to occur at a half of the overall imaging duration of the first heart imaging.

One form of embodiment makes provision, in the first heart imaging, for an evaluation of the first diagnostic measurement data and second diagnostic measurement data after the end of the imaging duration of the first heart imaging to have a duration that amounts to more than a quarter of the imaging duration.

One form of embodiment makes provision, in the first heart imaging, for a compressed sensing acceleration technique to be used in the first diagnostic recording and the second diagnostic recording.

One form of embodiment makes provision for the diagnostic measurement data recorded in the first heart imaging for assessing a heart function of the examination object.

One form of embodiment makes provision for the heart imaging to be a second heart imaging and for the number of diagnostic recordings exclusively to comprise the following diagnostic recordings:

A first diagnostic recording, which is embodied as a dynamic heart recording along long axis measurement slices of the heart,

A second diagnostic recording, which is embodied as a T1 mapping measurement,

A third diagnostic recording, which is embodied as a delayed enhancement measurement, and

A fourth diagnostic recording, which is embodied as a dynamic heart recording along short axis measurement slices of the heart.

One form of embodiment makes provision for a second maximum imaging duration to be predetermined, which amounts to a maximum of 18 minutes, for the second heart imaging.

One form of embodiment makes provision for the second maximum imaging duration to amount to a maximum of 10 minutes.

One form of embodiment makes provision, in the second heart imaging, for the second diagnostic recording and the third diagnostic recording to be carried out in the time between the first diagnostic recording and the fourth diagnostic recording.

One form of embodiment makes provision, in the second heart imaging, for there to be an application of contrast medium before the start of a first measurement block.

One form of embodiment makes provision, in the second heart imaging, for at least 10 minutes to elapse between the time of the application of contrast medium and the beginning of the third diagnostic recording.

One form of embodiment makes provision, in the second heart imaging, for the first diagnostic recording and the second diagnostic recording to be carried out in the time before the third diagnostic recording and for the fourth diagnostic recording to be carried out in the time after the third diagnostic recording.

One form of embodiment makes provision for the fourth diagnostic recording to be placed in the second heart imaging such that a contrast medium accumulation in the heart of the examination object is already reduced again by the time of the fourth diagnostic recording.

One form of embodiment makes provision, in the second heart imaging, for the measurement blocks with the overview recordings, totaled up, to have a duration that is shorter than the totaled-up duration of the measurement blocks with the diagnostic recordings.

One form of embodiment makes provision for the diagnostic measurement data recorded in the second heart imaging to be embodied for assessing a heart function and the possible presence of a non ischemic cardiomyopathy of the examination object.

One form of embodiment makes provision for the heart imaging to be a third heart imaging and for the number of diagnostic recordings exclusively to comprise the following diagnostic recordings:

A first diagnostic recording, which is embodied as a dynamic heart recording along long axis measurement slices of the heart,

A second diagnostic recording, which is embodied as a perfusion measurement,

A fourth diagnostic recording, which is embodied as a T1 mapping measurement,

A fifth diagnostic recording, which is embodied as a dynamic heart recording along short axis measurement slices of the heart, and

A sixth diagnostic recording, which is embodied as a delayed enhancement measurement.

One form of embodiment makes provision for a second maximum imaging duration, which amounts to a maximum of 22 minutes, to be predetermined for the third heart imaging.

One form of embodiment makes provision for the third maximum imaging duration to amount to a maximum of 15 minutes.

One form of embodiment makes provision, in the third heart imaging, for there to be an application of contrast medium in the time after the first diagnostic recording and in the time before the second diagnostic recording.

One form of embodiment makes provision, in the third heart imaging, for at least 6 minutes to elapse between the time of the application of contrast medium and the beginning of the sixth diagnostic recording.

One form of embodiment makes provision, in the third heart imaging, for the fourth diagnostic recording and the fifth diagnostic recording to occur in the time between the second diagnostic recording and the sixth diagnostic recording.

One form of embodiment makes provision for there additionally to be a third diagnostic recording in the time between the second diagnostic recording and the sixth diagnostic recording, which is embodied as a thorax recording in the coronal and/or transversal measurement slices.

One form of embodiment makes provision, in the third heart imaging, for the measurement blocks with overview recordings, totaled up, to have a duration that is shorter than the totaled-up duration of the measurement blocks with the diagnostic recordings.

One form of embodiment makes provision for the diagnostic measurement data for assessing a heart function recorded in the third heart imaging to be embodied for assessing the possible presence of a non ischemic cardiomyopathy of the examination object and the possible presence of an ischemic cardiomyopathy of the examination object.

The proposed execution sequences for heart imaging can offer the advantage that image data with a very good image quality can be recorded from the heart of the examination object. In this way, on the basis of the acquired image data, a heart function and/or a non ischemic cardiomyopathy and/or an ischemic cardiomyopathy can be investigated. Naturally other indications appearing sensible to be person skilled in the art can also be investigated on the basis of the acquired image data. In this way for example a proportion of inactive tissue or scar tissue in the myocard can be determined especially advantageously. Also, as an alternative or in addition, further tissue properties of the myocard tissue can be established. An evaluation of a reduced heart function and/or of a cardiomyopathy can likewise be possible.

It is precisely a possible integrated evaluation of the acquired measurement data (so-called inline processing) that can lead to a shortening of a period of time until final examination results and/or examination reports are available. The integrated evaluation of the acquired measurement data for creation of diagnostic information, such as function parameters of the heart of the examination object for example, can take place in such cases entirely after the conclusion of the acquisition of all measurement data. As an alternative it is also conceivable for diagnostic measurement data to already be being reconstructed and/or evaluated, while the acquisition of further measurement data of the examination object is still going on. The integrated evaluation of the acquired measurement data, in addition to the purpose of creating the diagnostic information, can also offer the opportunity of defining dynamic recording parameters during the execution sequence of the heart imaging of the examination object. In addition an integrated evaluation of measurement data of the examination object acquired during a measurement block can be used for defining recording parameters, such as for example a positioning of measurement slices and/or a size of a recording region, for the acquisition of measurement data of the examination object in a following measurement block. Thus the integrated evaluation of the acquired measurement data can fulfill a valuable double function.

Furthermore the proposed heart imaging can offer the advantage that the image data of the heart of the examination object, needed for a specific diagnostic issue, can be recorded especially quickly. At the same time there can be especially few movement artifacts present in the acquired image data. In this way the proposed heart imaging can advantageously also be used for examination objects that are not behaving cooperatively and/or cannot hold their breath for a long period and/or have an irregular heartbeat. The acquired image data can also be post-processed at a speed such that desired evaluation results of the image data are available a maximum of five minutes, advantageously a maximum of three minutes, highly advantageously a maximum of 90 seconds after the conclusion of the carrying out of the heart imaging.

Furthermore the proposed heart imaging can offer the advantage of being especially user-friendly and easy to operate. It is advantageously conceivable for the proposed heart imaging also to be carried out by personnel without any particular training. Here above all the proposed automations in the execution sequence of the heart imaging and/or the proposed minimization of any user interaction needed during the heart imaging can also make the acquisition of high-quality image data possible for an inexperienced user. Also a standardized execution sequence of the proposed heart imaging can lead to consistent investigation results with good comparability.

At least one embodiment of the inventive magnetic resonance device comprises a measurement data acquisition unit and a processing unit, wherein the magnetic resonance device is designed to carry out at least one embodiment of an inventive method.

Thus the processing unit in particular is embodied to carry out computer-readable instructions, in order to execute at least one embodiment of the inventive method. In particular the magnetic resonance device comprises a memory unit, wherein computer-readable information is stored in the memory unit, wherein the processing unit is embodied to load the computer-readable information from the memory unit and to execute the computer-readable information, in order to carry out at least one embodiment of an inventive method.

The processing unit can be embodied to send control signals to the magnetic resonance device, in particular to the measurement data acquisition unit of the magnetic resonance device, and/or to receive and/or to process control signals in order to carry out at least one embodiment of an inventive method. The processing unit can be integrated into the magnetic resonance device. The processing unit can also be installed separately from the magnetic resonance device. The processing unit can be connected to the magnetic resonance device.

For support when carrying out at least one embodiment of the inventive method, the processing unit can be embodied in a number of sub-processing units, which provide support during the execution of different tasks for the heart imaging or which carry out these different tasks.

Thus a first sub-processing unit of the processing unit can be embodied as a host processor. The host processor is embodied in particular for preparing and processing the user interactions. The host processor can further be embodied for activating the magnetic resonance device for carrying out the heart imaging. Furthermore the host processor can already be further processing reconstructed image data in the overview recordings and diagnostic recordings. The further processing of the image data by the host processor can for example comprise an evaluation of the image data, for example an establishment of the function parameters of the heart. As an alternative or in addition, the further processing of the image data by the host processor can also comprise a calculation of recording parameters for following measurements on the basis of the image data.

A second sub-processing unit of the processing unit can be embodied as a reconstruction processor. The reconstruction processor is embodied in particular for reconstruction of image data from the overview measurement data and diagnostic measurement data. For this the reconstruction processor can be exchanging data with the host processor. The reconstruction processor can be integrated in particular into the magnetic resonance device. The reconstruction processor can already be reconstructing acquired measurement data in parallel to the acquisition of further measurement data. In this way reconstructed image data for further processing by the host processor can already be available while the heart imaging is being carried out in the sense of “inline processing”. Also the reconstruction processor can take on part of the further processing of the reconstructed image data, in particular for processing recording parameters for following measurements. In this way the reconstruction processor can be embodied for example to recognize landmarks in image data for automatic determination of a recording region.

The components of the processing unit of at least one embodiment of the inventive magnetic resonance device can be preponderantly embodied in the form of software components. Basically however these components can also be realized partly in the form of software-supported hardware components, in particular where especially fast processing is involved, for example FPGAs or the like. Likewise the interfaces needed, for example when only an acceptance of data from other software components is involved, can be embodied as software interfaces. They can however also be embodied as interfaces constructed from hardware, which will be activated by suitable software. Of course it is also conceivable for a number of the the components to be realized grouped together in the form of an individual software component or software-supported hardware components.

Thus the magnetic resonance device, in particular the measurement data acquisition unit and the processing unit, is embodied to carry out a method for recording diagnostic measurement data of a heart of an examination object in a heart imaging with the following methods:

Carrying out a number of overview recordings of the heart of the examination object, wherein overview measurement data is acquired in the number of overview recordings, and

Carrying out a number of diagnostic recordings of the heart of the examination object based on the acquired overview measurement data, wherein diagnostic measurement data is acquired in the number of diagnostic recordings.

In accordance with one form of embodiment, the magnetic resonance device, in particular the measurement data acquisition unit and the processing unit, is embodied such that the at least two overview recordings and the at least two diagnostic recordings are carried out in their temporal execution sequence at least partly nested in one another.

In accordance with one form of embodiment, the magnetic resonance device, in particular the measurement data acquisition unit and the processing unit, is embodied such that, in the heart imaging, before the temporally first diagnostic recording of the number of diagnostic recordings, there are more than twice as many overview recordings as there are overview recordings between the temporally first diagnostic recording of the number of diagnostic recordings and the temporally second diagnostic recording of the number of diagnostic recordings.

In accordance with one form of embodiment, the magnetic resonance device, in particular the measurement data acquisition unit and the processing unit, is embodied such that the figure for the number of overview recordings amounts to a maximum of six.

In accordance with one form of embodiment, the magnetic resonance device, in particular the measurement data acquisition unit and the processing unit, is embodied such that the temporally first diagnostic recording of the number of diagnostic recordings and the temporally second diagnostic recording of the number of diagnostic recordings are carried out along different heart axes of the examination object.

In accordance with one form of embodiment, the magnetic resonance device, in particular the measurement data acquisition unit and the processing unit, is embodied such that measurement slices orthogonal to one another are acquired in the heart of the examination object in the temporally first diagnostic recording of the number of diagnostic recordings and measurement slices in parallel to one another are acquired in the heart of the examination object in the temporally second diagnostic recording of the number of diagnostic recordings.

In accordance with one form of embodiment, the magnetic resonance device, in particular the measurement data acquisition unit and the processing unit, is embodied so that a planning of the measurement slices in parallel to one another is based on the measurement slices orthogonal to one another acquired in the temporally first diagnostic recording.

In accordance with one form of embodiment, the magnetic resonance device, in particular the measurement data acquisition unit and the processing unit, is embodied so that there are a number of measurement blocks with overview recordings before the beginning of a measurement block with the temporally first diagnostic recording of the number of diagnostic recordings, wherein the number of measurement blocks with the overview recordings, totaled up, last more than twice as long as the measurement block with the temporally first diagnostic recording.

In accordance with one form of embodiment, the magnetic resonance device, in particular the measurement data acquisition unit and the processing unit, is embodied so that, at the beginning of the heart imaging, there is at least one overview measurement for positioning the heart in an isocenter of the magnetic resonance device and at least one overview measurement for defining an orientation and/or a recording region of long axis measurement slices.

In accordance with one form of embodiment, the magnetic resonance device, in particular the measurement data acquisition unit and the processing unit, is embodied so that the at least one measurement block with the at least one overview measurement for defining the orientation and/or the recording region of long axis measurement slices lasts for a longer time than the at least one measurement block with the at least one overview measurement for positioning the heart in the isocenter of the magnetic resonance device.

In accordance with one form of embodiment, the magnetic resonance device, in particular the measurement data acquisition unit and the processing unit, is embodied so that the carrying out of at least one part of the number of diagnostic recordings comprises the use of a compressed sensing acceleration technique.

In accordance with one form of embodiment, the magnetic resonance device, in particular the measurement data acquisition unit and the processing unit, is embodied so that there are a maximum of five user interactions during the heart imaging.

In accordance with one form of embodiment, the magnetic resonance device, in particular the measurement data acquisition unit and the processing unit, is embodied so that a combined figure for the number of overview recordings and the number of diagnostic recordings is at least twice as large as a figure for the number of user actions that take place during the heart imaging.

In accordance with one form of embodiment, the magnetic resonance device, in particular the measurement data acquisition unit and the processing unit, is embodied so that precisely one user interaction takes place between the temporally first diagnostic recording of the number of diagnostic recordings and the temporally second diagnostic recording of the number of diagnostic recordings.

In accordance with one form of embodiment, the magnetic resonance device, in particular the measurement data acquisition unit and the processing unit, is embodied so that there are at least twice as many user interactions before the beginning of the temporally first diagnostic recording of the number of diagnostic recordings as there are user interactions between the temporally first diagnostic recording and the temporally second diagnostic recording of the number of diagnostic recordings.

In accordance with one form of embodiment, the magnetic resonance device, in particular the measurement data acquisition unit and the processing unit, is embodied so that there are more automatic evaluation steps than user interactions during the heart imaging.

In accordance with one form of embodiment, the magnetic resonance device, in particular the measurement data acquisition unit and the processing unit, is embodied so that, for a user interaction needed, the user is automatically presented with suggestions, which will simply be accepted or modified by the user for the user interaction.

In accordance with one form of embodiment, the magnetic resonance device, in particular the measurement data acquisition unit and the processing unit, is embodied so that for a user interaction needed, the user is automatically provided at a display unit with instructions for the user interaction and/or with suitable tools for the user interaction.

In accordance with one form of embodiment, the magnetic resonance device, in particular the measurement data acquisition unit and the processing unit, is embodied so that a maximum imaging duration is predetermined for the heart imaging, wherein imaging parameters for the heart imaging are only able to be set by a user such that the maximum imaging duration is not exceeded with the set imaging parameters.

In accordance with one form of embodiment, the magnetic resonance device, in particular the measurement data acquisition unit and the processing unit, is embodied so that the heart imaging is a first heart imaging and the number of diagnostic recordings exclusively comprise the following diagnostic recordings:

A first diagnostic recording, which is embodied as a dynamic heart recording along long axis measurement slices of the heart, and

A second diagnostic recording, which is embodied as a dynamic heart recording along short axis measurement slices of the heart.

In accordance with one form of embodiment, the magnetic resonance device, in particular the measurement data acquisition unit and the processing unit, is embodied so that a first maximum imaging duration, which amounts to a maximum of 12 minutes, is predetermined for the first heart imaging.

In accordance with one form of embodiment, the magnetic resonance device, in particular the measurement data acquisition unit and the processing unit, is embodied so that the first maximum imaging duration amounts to a maximum of 6 minutes.

In accordance with one form of embodiment, the magnetic resonance device, in particular the measurement data acquisition unit and the processing unit, is embodied so that, in the first heart imaging, the second diagnostic recording follows on in time from the first diagnostic recording.

In accordance with one form of embodiment, the magnetic resonance device, in particular the measurement data acquisition unit and the processing unit, is embodied so that, in the first heart imaging, the short axis measurement slices are planned based on the diagnostic measurement data acquired in the first diagnostic recording.

In accordance with one form of embodiment, the magnetic resonance device, in particular the measurement data acquisition unit and the processing unit, is embodied so that, in the first heart imaging, more than twice as many short axis measurement slices are acquired in the second diagnostic recording as there are long axis measurement slices acquired in the first diagnostic recording.

In accordance with one form of embodiment, the magnetic resonance device, in particular the measurement data acquisition unit and the processing unit, is embodied so that, in the first heart imaging, a figure for the number of overview recordings is at least twice as large as a figure for the number of the diagnostic recordings.

In accordance with one form of embodiment, the magnetic resonance device, in particular the measurement data acquisition unit and the processing unit, is embodied so that the first heart imaging is carried out without application of contrast medium.

In accordance with one form of embodiment, the magnetic resonance device, in particular the measurement data acquisition unit and the processing unit, is embodied so that, in the first heart imaging, the measurement block with the second diagnostic recording has a shorter duration than the measurement block with the first diagnostic recording.

In accordance with one form of embodiment, the magnetic resonance device, in particular the measurement data acquisition unit and the processing unit, is embodied so that, in the first heart imaging, the measurement blocks with the overview recordings, totaled up, need a longer duration than the totaled-up measurement blocks with the diagnostic recordings.

In accordance with one form of embodiment, the magnetic resonance device, in particular the measurement data acquisition unit and the processing unit, is embodied so that the start of the measurement block with the first diagnostic recording occurs at a half of the overall imaging duration of the first heart imaging.

In accordance with one form of embodiment, the magnetic resonance device, in particular the measurement data acquisition unit and the processing unit, is embodied so that, during the first heart imaging, an evaluation of the first diagnostic measurement data and second diagnostic measurement data after the end of the imaging duration of the first heart imaging has a duration that amounts to more than a quarter of the imaging duration.

In accordance with one form of embodiment, the magnetic resonance device, in particular the measurement data acquisition unit and the processing unit, is embodied so that, in the first heart imaging, a compressed sensing acceleration technique is used for the first diagnostic recording and the second diagnostic recording.

In accordance with one form of embodiment, the magnetic resonance device, in particular the measurement data acquisition unit and the processing unit, is embodied so that the diagnostic measurement data recorded in the first heart imaging is used for assessing a heart function of the examination object

In accordance with one form of embodiment, the magnetic resonance device, in particular the measurement data acquisition unit and the processing unit, is embodied so that the heart imaging is a second heart imaging and the number of diagnostic recordings exclusively comprise the following diagnostic recordings:

A first diagnostic recording, which is embodied as a dynamic heart recording along long axis measurement slices of the heart,

A second diagnostic recording, which is embodied as a T1-mapping measurement,

A third diagnostic recording, which is embodied as a delayed enhancement measurement, and

A fourth diagnostic recording, which is embodied as a dynamic heart recording along short axis measurement slices of the heart.

In accordance with one form of embodiment, the magnetic resonance device, in particular the measurement data acquisition unit and the processing unit, is embodied so that a second maximum imaging duration, which amounts to a maximum of 18 minutes, is predetermined for the second heart imaging.

In accordance with one form of embodiment, the magnetic resonance device, in particular the measurement data acquisition unit and the processing unit, is embodied so that the second maximum imaging duration amounts to a maximum of 10 minutes.

In accordance with one form of embodiment, the magnetic resonance device, in particular the measurement data acquisition unit and the processing unit, is embodied so that, in the second heart imaging, the second diagnostic recording and the third diagnostic recording occur in the time between the first diagnostic recording and the fourth diagnostic recording.

In accordance with one form of embodiment, the magnetic resonance device, in particular the measurement data acquisition unit and the processing unit, is embodied so that, in the second heart imaging there is an application of contrast medium before the start of a first measurement block.

In accordance with one form of embodiment, the magnetic resonance device, in particular the measurement data acquisition unit and the processing unit, is embodied so that, in the second heart imaging, at least 10 minutes elapse between the time of the application of contrast medium and the beginning of the third diagnostic recording.

In accordance with one form of embodiment, the magnetic resonance device, in particular the measurement data acquisition unit and the processing unit, is embodied so that, in the second heart imaging, the first diagnostic recording and the second diagnostic recording are carried out in the time before the third diagnostic recording and the fourth diagnostic recording is carried out in the time after the third diagnostic recording.

In accordance with one form of embodiment, the magnetic resonance device, in particular the measurement data acquisition unit and the processing unit, is embodied so that the fourth diagnostic recording is placed in the second heart imaging such that a contrast medium accumulation in the heart of the examination object is already reduced again at the time of the fourth diagnostic recording.

In accordance with one form of embodiment, the magnetic resonance device, in particular the measurement data acquisition unit and the processing unit, is embodied so that, in the second heart imaging, the measurement blocks with the overview recordings, totaled up, have a duration that is shorter than the totaled-up duration of the measurement blocks with the diagnostic recordings.

In accordance with one form of embodiment, the magnetic resonance device, in particular the measurement data acquisition unit and the processing unit, is embodied so that the diagnostic measurement data recorded in the second heart imaging is embodied for assessing a heart function and the possible presence of a non ischemic cardiomyopathy of the examination object.

In accordance with one form of embodiment, the magnetic resonance device, in particular the measurement data acquisition unit and the processing unit, is embodied so that the heart imaging is a third heart imaging and the number of diagnostic recordings exclusively comprise the following diagnostic recordings:

A first diagnostic recording, which is embodied as a dynamic heart recording along long axis measurement slices of the heart,

A second diagnostic recording, which is embodied as a perfusion measurement,

A fourth diagnostic recording, which is embodied as a T1-mapping measurement,

A fifth diagnostic recording, which is embodied as a dynamic heart recording along short axis measurement slices of the heart, and

A sixth diagnostic recording, which is embodied as a delayed enhancement measurement.

In accordance with one form of embodiment, the magnetic resonance device, in particular the measurement data acquisition unit and the processing unit, is embodied so that, for the third heart imaging, a second maximum imaging duration is predetermined, which amounts to a maximum of 22 minutes.

In accordance with one form of embodiment, the magnetic resonance device, in particular the measurement data acquisition unit and the processing unit, is embodied so that the third maximum imaging duration amounts to a maximum of 15 minutes.

In accordance with one form of embodiment, the magnetic resonance device, in particular the measurement data acquisition unit and the processing unit, is embodied so that, in the third heart imaging, there is an application of contrast medium in the time after the first diagnostic recording and in the time before the second diagnostic recording.

In accordance with one form of embodiment, the magnetic resonance device, in particular the measurement data acquisition unit and the processing unit, is embodied so that, in the third heart imaging, at least 6 minutes elapse between the time of the application of contrast medium and the beginning of the sixth diagnostic recording.

In accordance with one form of embodiment, the magnetic resonance device, in particular the measurement data acquisition unit and the processing unit, is embodied so that, in the third heart imaging, the fourth diagnostic recording and the fifth diagnostic recording occur in the time between the second diagnostic recording and the sixth diagnostic recording.

In accordance with one form of embodiment, the magnetic resonance device, in particular the measurement data acquisition unit and the processing unit, is embodied so that a third diagnostic recording, which is embodied as a thorax recording in coronal and/or transversal measurement slices, occurs additionally in the time between the second diagnostic recording and the sixth diagnostic recording.

In accordance with one form of embodiment, the magnetic resonance device, in particular the measurement data acquisition unit and the processing unit, is embodied so that, in the third heart imaging, the measurement blocks with the overview recordings, totaled up, have a duration that is shorter than the totaled-up duration of the measurement blocks with the diagnostic recordings.

In accordance with one form of embodiment, the magnetic resonance device, in particular the measurement data acquisition unit and the processing unit, is embodied so that the diagnostic measurement data recorded in the third heart imaging is embodied for assessing a heart function, the possible presence of a non ischemic cardiomyopathy and the possible presence of an ischemic cardiomyopathy of the examination object.

At least one embodiment of the inventive computer program product is able to be loaded directly into a memory of a programmable processing unit of a magnetic resonance device and has program code segments/modules for carrying out at least one embodiment of an inventive method, when the computer program product is executed in the processing unit of the magnetic resonance device. The computer program product can be a computer program or can include a computer program. This enables at least one embodiment of the inventive method to be carried out quickly, in an identically repeatable manner and robustly.

The computer program product is configured so that it can execute at least one embodiment of the inventive method steps via the processing unit. The processing unit in such cases must have the respective prerequisites in each case, such as a corresponding main memory, a corresponding graphics card or a corresponding logic unit, so that the respective method steps can be carried out efficiently.

In at least one embodiment, the computer program product is stored for example on a computer-readable medium or is held on a server or a network, from where it can be loaded into the processor of a local processing unit, which is directly connected to the magnetic resonance device or can be embodied as part of the magnetic resonance device. Furthermore control information of the computer program product can be stored on an electronically-readable data medium. The control information of the electronically-readable data medium can be designed so that, when the data medium is used in a processing unit of the magnetic resonance device, it carries out at least one embodiment of an inventive method. Thus the computer program product can also represent an electronically-readable data medium.

Examples of electronically-readable data media are a DVD, a magnetic tape, a hard disk or a USB stick, on which electronically-readable control information, in particular software (cf. above), is stored. When this control information (software) is read from the data medium and stored in a controller and/or processing unit of the magnetic resonance device, all inventive forms of embodiment of the previously described method can be carried out. Thus the invention can also be based on the the computer-readable medium and/or the the electronically-readable data medium.

The advantages of the inventive magnetic resonance device and of the inventive computer program products essentially correspond to the advantages of the inventive method, which have been set out in detail above. Features, advantages or alternate forms of embodiment mentioned here are likewise also to be transferred to the other claimed subject matter and vice versa. In other words the device claims can also be further developed with the features that are described or claimed in conjunction with a method. The corresponding functional features of the method are embodied in such cases by corresponding physical modules, in particular by hardware modules.

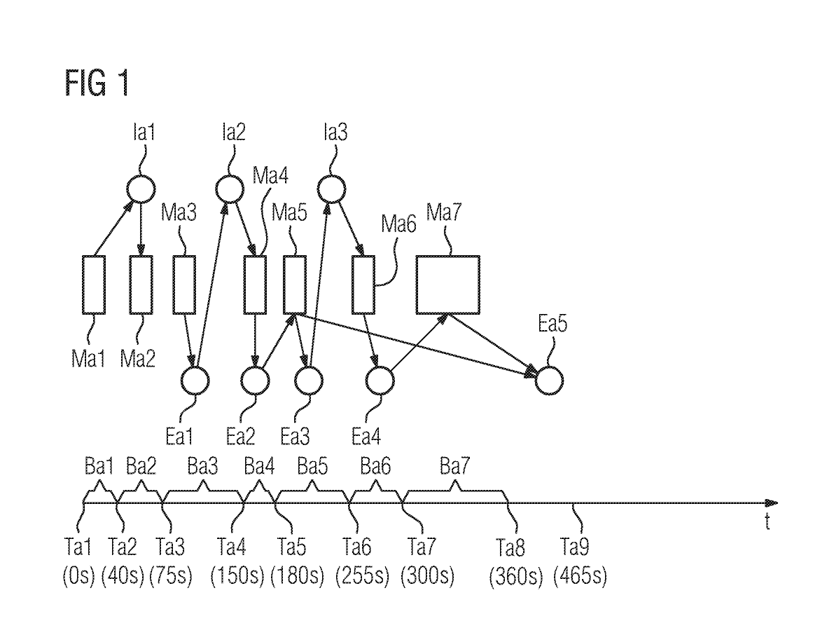

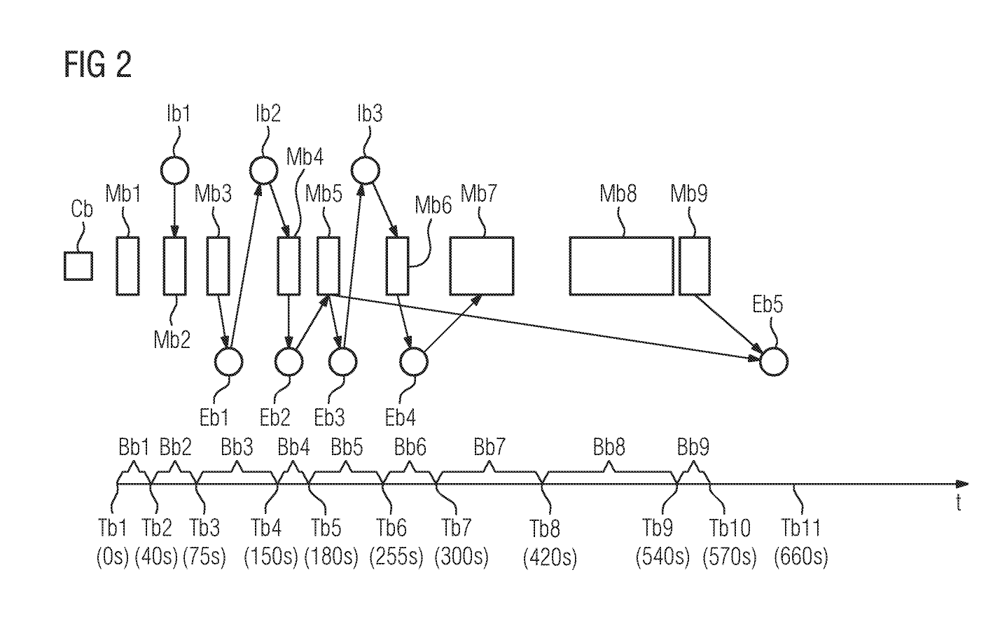

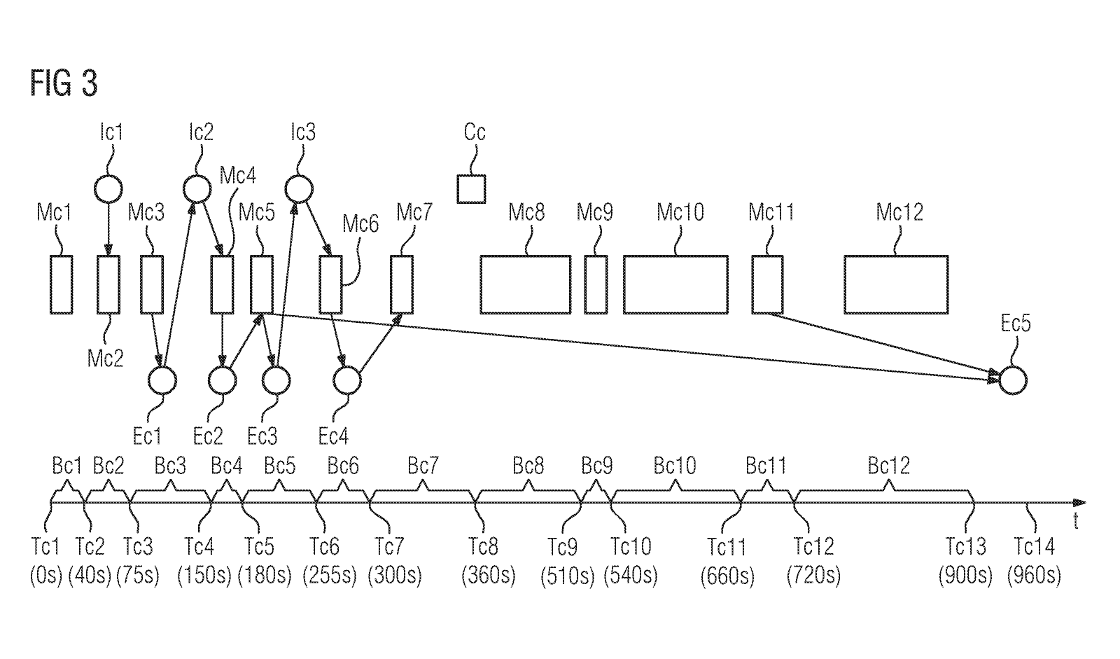

Three possible execution sequences of heart imagings are shown in FIGS. 1-3. Thus an execution sequence of a first heart imaging is shown in FIG. 1. FIG. 2 shows an execution sequence of a second heart imaging. The execution sequence of a third heart imaging is explained in FIG. 3. In the respective description for the figures, first of all, for each heart imaging, the concrete execution sequence or workflow for the respective heart imaging is described. Subsequently different acceleration techniques and automation techniques are explained for the respective heart imaging.

The heart imagings presented in FIGS. 1-3 in particular each represent a measurement session, in which the examination object is examined via the magnetic resonance device. In this way the examination object in particular remains positioned in the magnetic resonance device during the complete execution sequence of a heart imaging shown.

The described heart imagings are each divided up into a number of, in particular, directly-consecutive measurement blocks Ba, Bb, Bc. In such cases there is in particular a recording Ma, Mb, Mc of measurement data in each measurement block Ba, Bb, Bc. A measurement block Ba, Bb, Bc can in such cases, as well as the recording Ma, Mb, Mc of the measurement data, comprise a user interaction for preparation of the recording Ma, Mb, Mc. In the user interaction the recording parameters for the recording Ma, Mb, Mc, which takes place in the measurement block Ba, Bb, Bc can be validated. The recording parameters can be defined on the basis of measurement data acquired in a preceding measurement block Ba, Bb, Bc. Furthermore the measurement block Ba, Bb, Bc can comprise a reconstruction and possibly a further evaluation of the measurement data acquired in the measurement block Ba, Bb, Bc.

In such cases the recording can be an overview recording, in which overview measurement data is acquired. The overview measurement data is primarily, possibly exclusively, intended in such cases for defining recording parameters of a recording Ma, Mb, Mc, which takes place in one of the following measurement blocks Ba, Bb, Bc. The overview measurement data is preferably used to define recording parameters for a measurement in a following measurement block Ba, Bb, Bc. Image data, which will be stored in a database, can also continue to be reconstructed from the overview measurement data. The image data reconstructed from the overview measurement data is however usually not of central interest for the diagnosis. The overview measurement data can also be stored together with the image data. As a rule overview measurement data will only be shown to a doctor during diagnosis to the extent that it shows them the point at which actual diagnostic image data has been recorded. Thus the position or the positions that identify the position of the actual diagnostic image data in the body can be indentified in the overview measurement data for example. In some cases it is also conceivable for the overview measurement data not to be stored in a database and to be discarded again after it has been used for defining the recording parameters.

As an alternative or in addition the recording Ma, Mb, Mc can be a diagnostic recording, in which diagnostic measurement data is acquired. Diagnostic image data in particular can be generated from the diagnostic measurement data, which can be displayed on a display unit to a doctor making the diagnosis. The diagnostic measurement data thus in particular represents such data as will be reconstructed into image data, which will be displayed to a doctor in a later diagnostic finding, in order to make the actual diagnosis on the basis of the image data. As an alternative or in addition physiological parameters of the heart of the examination object can be computed from the diagnostic measurement data, which can be provided to the doctor making the diagnosis. In addition the diagnostic measurement data can also be used to define recording parameters of a recording Ma, Mb, Mc, which is made in one of the following measurement blocks Ba, Bb, Bc.

The measurement blocks Ba, Bb, Bc can additionally also comprise an evaluation step Ea, Eb, Ec, in which the measurement data acquired during the respective measurement block Ba, Bb, Bc is evaluated. The measurement data is evaluated in evaluation step Ea, Eb, Ec in particular immediately after the acquisition of the measurement data. The evaluation of the measurement data in the evaluation step Ea, Eb, Ec in such cases typically delivers information for defining recording parameters of a recording Ma, Mb, Mc, which is made in one of the following measurement blocks Ba, Bb, Bc. Before the definition of the recording parameters a reconstruction will have typically already been carried out of, in particular time-resolved, image data from the diagnostic measurement data, wherein the recording parameters can then be defined on the basis of the image data. In this way in particular the same image data that is displayed to a doctor for diagnosis, is also used for defining the recording parameters. As an alternative the measurement data can also only be reconstructed to such an extent in the evaluation step Ea, Eb, Ec, so that, on the basis of the reconstructed image data, only a definition of the recording parameters of a recording that is made in one of the following measurement blocks is possible.

The recording parameters can be established automatically in such cases by an, in particular algorithmic, evaluation of overview image data that has been reconstructed from the acquired overview measurement data. If, in evaluation step Ea, Eb, Ec there is an evaluation of overview measurement data for definition of recording parameters for a measurement in a following measurement block Ba, Bb, Bc, then this evaluation step Ea, Eb, Ec can require an especially short duration. Overview image data reconstructed from the overview measurement data can be reconstructed in a fraction of the time of the associated measurement block and can be displayed to a user at a user interface, for example for validating the determination of the recording parameters.

In addition the measurement blocks Ba, Bb, Bc can also comprise a user interaction Ia, Ib, Ic. In the user interaction Ia, Ib, Ic there is in particular an input of a command of a user via a suitable input unit. In such cases recording parameters for the recording Ma, Mb, Mc in the respective measurement block Ba, Bb, Bc and/or for a following recording Ma, Mb, Mc can be entered in such cases in the user interaction Ia, Ib, Ic. The user interaction Ia, Ib, Ic can also comprise a validation, which in particular comprises a check, of automatically established recording parameters. Of course recording parameters can also be changed in the user action Ia, Ib, Ic.

The presentation of the heart imagings in FIGS. 1-3 is in this case always embodied along a horizontal time line t, which is arranged on the lower edge of the figures. A number of points in time Ta, Tb, Tc are indicated on the time line in each case. The points in time form start and end times of measurement blocks Ba, Bb, Bc, the duration in time and arrangement of which is indicated directly above the horizontal time line. For each measurement block Ba, Bb, Bc the respective recording Ma, Mb, Mc is indicated as a small box. Halt points for the duration in time of the recordings Ma, Mb, Mc and the positionings of the recordings Ma, Mb, Mc within the respective measurement block Ba, Bb, Bc can be read off in this case from FIGS. 1-3. However durations in time of the recordings Ma, Mb, Mc differing from the diagram and different positionings of the recordings Ma, Mb, Mc are of course also conceivable within the respective measurement block Ba, Bb, Bc.

User interactions Ia, Ib, Ic possibly occurring in the measurement block Ba, Bb, Bc are indicated as a circle above the recordings Ma, Mb, Mc. Evaluation steps Ea, Eb, Ec possibly occurring in the measurement block Ba, Bb, Bc are indicated as a circle below the recordings Ma, Mb, Mc. The user interactions Ia, Ib, Ic and evaluation steps Ea, Eb, Ec are indicated in this case at their typical time position with an example duration within the heart imaging. Halt points for the temporal positionings of the user interactions Ia, Ib, Ic and evaluation steps Ea, Eb, Ec within the respective measurement block Ba, Bb, Bc can be read off in this case from FIGS. 1-3. However temporal positionings differing from the diagram and durations in time of the user interactions Ia, Ib, Ic and evaluation steps Ea, Eb, Ec are however also conceivable within the respective measurement block Ba, Bb, Bc.

FIG. 1—First Heart Imaging

General Information Relating to First Heart Imaging

The first heart imaging, the execution sequence of which is shown in FIG. 1, in particular delivers diagnostic measurement data that can serve as the basis for the evaluation of a heart function of the examination object. Preferably in this case similar diagnostic parameters of the heart of the examination object to those in an ultrasound measurement can be established in the first heart imaging. In this case it is in particular an aim of the first heart imaging to record the diagnostic measurement data needed for evaluating the heart function of the examination object in a first imaging duration that is as short as possible. The diagnostic measurement data in this case is preferably recorded in the shortest possible first imaging duration such that diagnostic parameters can be established and provided for determining the function of the heart of the examination object, such as for example an ejection fraction, a beat volume, a heart mass etc., in sufficient quality despite the comparatively short first imaging duration.

The first heart imaging has a first imaging duration, which lasts from a start time Ta1 of the first heart imaging to an eighth point in time Ta8, at which the recording of measurement data in the first heart imaging is ended. The first imaging duration preferably amounts in this case to a maximum of 12 minutes, advantageously to a maximum of 10 minutes, especially advantageously to a maximum of 8 minutes, highly advantageously to a maximum of 6 minutes. The first imaging duration is in particular embodied as the maximum imaging duration, which may not be exceeded when carrying out the first heart imaging. The first imaging duration can include a duration of user interactions or parameter settings for the acquisition of the measurement data. In specific cases it is also conceivable for the duration of a patient positioning to be calculated into the first imaging duration. As an alternative the first imaging duration can also be characterized by more than 60 percent, in particular more than 75 percent, highly advantageously more than 90 percent of a series of several examinations, which according to the scheme presented in FIG. 1 are carried out for the first heart imaging, to adhere to the first imaging duration.

In this case the especially advantageous case is shown in FIG. 1, in which the first imaging duration of the first heart imaging lasts 6 minutes. After conclusion of the recording of the measurement data in the first heart imaging further time can elapse, in which there is a post-processing and/or evaluation of the measurement data.

Description of a Possible Concrete Execution Sequence of the First Heart Imaging

Preparation of the First Heart Imaging

First of all it is defined in particular that a heart imaging of the examination object is to be carried out. Here a maximum imaging duration of the first heart imaging can be defined, wherein the maximum imaging duration may in particular not be exceeded by the first imaging duration. The maximum of the imaging duration can be defined directly, for example by a user entering the maximum imaging duration for the entire examination execution sequence of the first heart imaging directly into an input mask. The maximum of the imaging duration can also be defined indirectly, for example by the user selecting from a plurality of defined, different execution sequences for the heart imaging, for example via an interaction at a user interface, a variant linked to the maximum imaging duration, in particular the first heart imaging.

Before the start time Ta1 of the first heart imaging patient-specific features can be acquired automatically or manually. Imaging parameters for the first heart imaging can then be adapted on the basis of the patient-specific features. The subsequent time sequence of the individual measurement blocks can be varied based on the concrete entry of the patient-specific feature and as a function thereof.

A possible patient-specific feature is a length of time for which the examination object, in particular a patient, can hold their breath, and/or information as to whether the examination object, in particular the patient, can hold their breath at all. On the basis of this patient-specific feature, periods of time of individual measurements and/or number of breathholds can then be adapted per measurement. A choice of protocols, which can be executed when breathing freely, can then be carried out. A further possible patient-specific feature is a language that is to be used for commands directed to the examination object. A further possible patient-specific feature is a choice of a trigger modality. In this way it can be determined for example whether an electrocardiogram (EKG) and/or a pulse meter is to be used for a determination of heart phases of the examination object. Furthermore a body size of the examination object can be acquired for example. On the basis of the body size a typical position of the heart of the examination object can be estimated, so that the heart of the examination object can already be positioned approximately in the isocenter of the magnetic resonance device.

After the acquisition of the patient-specific features and a suitable positioning of the patient support facility, on which the examination object is supported, in the magnetic resonance device, the first heart imaging can be started. The first heart imaging starts in this case in particular after actuation of a start button by a user. The first heart imaging can also start automatically after conclusion of the preparations.

Measurement Block Ba1

The first heart imaging shown starts at the first point in time Ta1 or start time Ta1 with a first measurement block Ba1. In the first measurement block Ba1 a first overview recording Ma1 is made, during which first overview measurement data is acquired.

The first measurement block Ba1, in the case shown, has a first duration of 40 s. Between 2 and 10 seconds, in particular between 4 and 8 seconds, in particular 6 seconds, of the first period of time are taken up by the pure measurement time of the first overview recording Ma1 for acquiring the first overview measurement data. Pure measurement time here refers in particular only to that time that is needed for the acquisition of the magnetic resonance signals that form the measurement. Thus the pure measurement time can merely comprise a time for filling the k space with the measurement data. A further duration of the first measurement block Ba1 can be taken up partly by a preparation of the acquisition of the first overview measurement data. The preparation of an acquisition of measurement data can for example comprise an output of speech commands to the examination object, for example to achieve a specific breathing position of the examination object. Furthermore adjustment measurements, which for example comprise an adaptation of a transmitter and receiver voltage of the magnetic resonance device, can count as preparation of the acquisition of the measurement data. The remaining duration of the first measurement block Ba1 can furthermore be taken up partly with an evaluation or post-processing of the first overview measurement data acquired during the first overview recording Ma1.

The first overview recording Ma1 is made of a thorax region of the examination object. The first overview recording Ma1 is thus in particular a measurement that is used for the definition of the recording parameters for subsequent measurement blocks. Usually it no longer plays any definite role for the further diagnosis of the diagnostic measurement data after the recording scheme shown in FIG. 1 has been carried out. Thus the first overview recording Ma1 can also be generally referred to as a localizer measurement or scout measurement. The overview measurement data acquired in the first overview recording Ma1 comprises in particular a number of low-resolution measurement slices, advantageously in different slice orientations.

The first overview recording Ma1 can be made when the examination object is holding their breath or when the examination object is breathing freely. If the first overview recording Ma1 is made when the examination object is holding their breath, then typically one breathhold is needed for the acquisition of the first overview measurement data.

The number of slices for the first overview recording Ma1 and the resolution, indirectly connected thereto the number of items of measurement data recorded, are in these cases typically selected or dimensioned such that the recording of all measurement data that is needed for the first overview recording Ma1 can be carried out in one breathhold process, i.e. typically within a maximum of 15 seconds.

On the basis of the first overview measurement data acquired in the first overview recording Ma1, a position of the heart of the examination object, in particular in a long direction of the examination object, can be identified. The position of the heart can be identified in this case manually, semi-automatically or automatically. On the basis of the identified position of the heart the patient support facility of the magnetic resonance device will be moved so that the heart of the examination object is positioned in the isocenter of the magnetic resonance device. This enables the second overview recording Ma2 in the following second measurement block Ba2 to be made of the heart of the examination object positioned in the isocenter.

This is done in the second measurement block Ba2 by way of a first user interaction Ia1. For this the first overview measurement data is displayed to a user on a display unit, in particular together with an indication of a position of the isocenter of the magnetic resonance device. Then, in the first user interaction Ia1, the user can position measurement slices for a second overview recording Ma2, which is made in the second measurement block Ba2. The measurement slices in this case are preferably positioned by the user such that the isocenter of the magnetic resonance device is arranged in the longitudinal direction at the height of the middle of the left ventricle of the heart of the examination object. Here the user can be guided by instructions displayed on the display unit, so that the user correctly carries out the positioning of the measurement slices for the second overview recording Ma2.

Overall, during the recording of the first overview measurement data in the first measurement block Ba1, the heart is not yet located explicitly in the isocenter (or only by chance), while a repositioning of the patient for the second measurement block Ba2 can be undertaken on the basis of the first overview measurement data, so that the heart lies more precisely at or closer to the isocenter during recording of the second overview measurement data of the second measurement block Ba2 than it does during the first measurement block Ba1.

Measurement Block Ba2

Following on from the first measurement block Ba1, at a second point in time Ta2, a second measurement block Ba2 starts during the first heart imaging. A second overview recording Ma2 is made in the second measurement block Ba2, during which second overview measurement data is acquired.

The second point in time Ta2 lies, in the case shown, 40 s after the start time Ta1 of the first heart imaging. The second measurement block Ba2, in the case shown, has a second duration of 35 s. Between 7 and 20 seconds, in particular between 11 and 17 seconds, in particular 14 seconds, of the second duration are taken up with the pure measurement time of the second overview recording Ma2 for acquiring the second overview measurement data. A remaining duration of the second measurement block Ba2 can be taken up partly by a preparation of the acquisition of the second overview measurement data, in particular in the first user interaction Ia1. The remaining duration of the second measurement block Ba2 can furthermore be taken up partly by an evaluation or post-processing of the second overview measurement data.

The second overview recording Ma2 is embodied as a localizer measurement or scout measurement, wherein the heart of the examination object is positioned in the isocenter of the magnetic resonance device. The overview measurement data acquired in the second overview recording Ma2 comprises in particular a number of low-resolution measurement slices, of which the position has been defined by the user in the first user interaction Ia1. The second overview measurement data too plays only a subordinate role after the definition of the recording parameters for the subsequent measurement blocks in the subsequent diagnostic examination by a doctor.

The second overview recording Ma2 can be made when the examination object is holding their breath or when the examination object is breathing freely. If the second overview recording Ma2 is made when the examination object is holding their breath, then typically one breathhold is needed for the acquisition of the second overview measurement data.

The number of slices for the second overview recording Ma2 and the resolution, indirectly connected thereto the number of items of measurement data recorded, are in these cases typically selected or dimensioned such that the recording of all measurement data that is needed for the second overview recording Ma2 can be carried out in one breathhold process, i.e. typically within a maximum of 15 seconds.

The third overview recording Ma3 in the third measurement block Ba3 can be carried out on the basis of the second overview measurement data acquired in the second overview recording Ma2.

It should be pointed out here that as an alternative to the diagram in FIG. 1, the first measurement block Ba1 and the second measurement block Ba2 can also be combined into one measurement block. Thus instead of the first overview recording Ma1 and the second overview recording Ma2, there can be just one overview recording, which as a result of an automatic positioning of the heart of the examination object in the isocenter or sufficiently close to the isocenter, already covers the heart of the examination object in a suitable way.

Measurement Block Ba3

Following on from the second measurement block Ba2, at a third point in time Ta3, a third measurement block Ba3 starts during the first heart imaging. A third overview recording Ma3 is made in the third measurement block Ba3, during which third overview measurement data is acquired.

The third point in time Ta3 lies, in the case shown, 75 s after the start time Ta1 of the first heart imaging. The third measurement block Ba3, in the case shown, has a third duration of 75 s. Between 13 and 29 seconds, in particular between 17 and 25 seconds, in particular 21 seconds, of the third duration are taken up by the pure measurement time of the third overview measurement Ma3 for acquiring the third overview measurement data. A remaining duration of the third measurement block Ba3 can be taken up partly by a preparation of the acquisition of the third overview measurement data. The remaining duration of the third measurement block Ba3 can furthermore be taken up partly by an evaluation or post-processing of the third overview measurement data, in particular in the first evaluation step Ea1 and in the second user interaction Ia2.

Before the beginning of the third overview recording Ma3 there can optionally be a user interaction not shown in FIG. 1, in which a measurement field for the third overview recording Ma3 is validated by the user. Here the user can preferably insure that the measurement slices of the third overview recording Ma3 cover the heart completely from the base of the heart to the tip of the heart. However this is not absolutely necessary. The user interaction directly before the beginning of the third overview recording Ma3 can also be dispensed with if algorithms are employed that evaluate the overview recording Ma2 fully automatically and position measurement slices such that, for the third overview recording Ma3, the heart is completely covered from the base of the heart to the tip of the heart.

The third overview measurement data acquired in the third overview recording Ma3 is embodied to define an orientation of long axis measurement slices, which run along the long axis (LAX) of the heart. In this way the third overview recording Ma3 can also be referred to as an auto-align localizer or auto-align scout.

The third overview recording Ma3 can be made when the examination object is holding their breath or when the examination object is breathing freely. If the third overview recording Ma3 is made when the examination object is holding their breath, then typically one breathhold is needed for the acquisition of the third overview measurement data.

The number of slices for the third overview recording Ma3 and the resolution, indirectly connected thereto the number of items of measurement data recorded, are in these cases typically selected or dimensioned such that the recording of all measurement data that is needed for the third overview recording Ma3 can be carried out in one breathhold process, i.e. typically within a maximum of 15 seconds.

A defined recording technique is used in particular for the third overview recording Ma3, so that the third overview measurement data is consistent with annotated Atlas measurement data from other examination objects. In addition a comparison of the third overview measurement data with Atlas measurement data in different breath states, such as for example inspiration or expiration, is also possible. Annotated Atlas measurement data from other examination objects can be stored in the system and be included for a comparison and the evaluation of the third overview recording.

In this way landmarks, which characterize defined points in the heart of the examination object, can be automatically identified on the basis of the third overview measurement data in a first evaluation step Ea1. Possible landmarks characterize at least one of the following points in the heart: The left atrium, the aortic root, the right ventricle, the left ventricle, the tip of the heart. The first evaluation step Ea1 further comprises that an automatic calculation of a position and orientation of the long axis measurement slices is carried out on the basis of the identified landmarks. These long axis measurement slices can then be acquired in the first diagnostic recording Ma5 in the fifth measurement block Ba5 acquired. For more precise information about identifying the long axis measurement slices the reader is referred to US 2012/0121152 A1, wherein its content is herewith fully included in this application and is hereby incorporated herein by reference, in its entirety.

The automatically established long axis measurement slices are validated by the user in a second user interaction Ia2. For this, image data of the heart of the examination object, on which the automatically identified long axis measurement slices are indicated, is displayed to the user, preferably on the display unit. The user can then check the long axis measurement slices and if necessary adapt their positioning and/or alignment manually. As an aid the user can already be shown preview images, which indicate an anatomy along the automatically identified long axis measurement slices.

For example—when a series of examinations is to be carried out, as is shown for the first heart imaging in accordance with FIG. 1—an algorithm with an accuracy of more than 70 percent, in particular more than 85 percent, highly advantageously more than 95 percent is used. This can mean that in clinical practice, on average in fewer than 50 percent, or in particular in fewer than 30 percent of the cases, must a user correct the automatically established long axis measurement slices and, in the overwhelming number of cases, can simply confirm and accept them.

Measurement Block Ba4

Following on from the third measurement block Ba3, at a fourth point in time Ta4, a fourth measurement block Ba4 starts during the first heart imaging. A fourth overview recording Ma4 is made in the fourth measurement block Ba4, during which fourth overview measurement data is acquired.

The fourth point in time Ta4 lies, in the case shown, 150 s after the start time Ta1 of the first heart imaging. The fourth measurement block Ba4, in the case shown, has a fourth duration of 30 s. Between 2 and 6 seconds, in particular between 3 and 5 seconds, in particular 4 seconds, of the fourth duration are taken up by the pure measurement time of the fourth overview measurement Ma4 for acquiring the fourth overview measurement data. A remaining duration of the fourth measurement block Ba4 can be taken up partly by a preparation of the acquisition of the fourth overview measurement data. The remaining duration of the fourth measurement block Ba4 can furthermore be taken up partly by an evaluation or post-processing of the fourth overview measurement data, in particular in the second evaluation step Ea2.

The fourth overview recording Ma4 can be referred to as a long axis localizer or long axis scout. The fourth overview recording Ma4 comprises a measurement of the long axis measurement slices, which are defined in the first evaluation step Ea1 on the basis of the third overview measurement data and have been validated in the second user interaction Ia2.

The fourth overview recording Ma4 can be made when the examination object is holding their breath or when the examination object is breathing freely. If the fourth overview recording Ma4 is made when the examination object is holding their breath, then typically one breathhold is needed for the acquisition of the fourth overview measurement data.

The number of slices for the fourth overview recording Ma4 and the resolution, indirectly connected thereto the number of items of measurement data recorded, are in these cases typically selected or dimensioned such that the recording of all measurement data that is needed for the fourth overview recording Ma4 can be carried out in one breathhold process, i.e. typically within a maximum of 15 seconds.

On the basis of the fourth overview measurement data acquired in the fourth overview recording Ma4, in a second evaluation step Ea2, a recording region along the long axis measurement slices is defined. The recording region is restricted in particular to an extent of the heart or of a chest cavity of the examination object along the long axis measurement slices. The recording region can be calculated automatically in this case, wherein typically no validation by the user is necessary. In specific cases it is also conceivable for there to be a user interaction not shown in FIG. 1, in which the recording region along the long axis measurement slices can be validated or adapted by the user. For more precise information about identifying the long axis measurement slices the reader is referred to US 2009/0290776 A1, wherein its content is herewith fully included in this application and is hereby incorporated herein by reference, in its entirety.

It is also conceivable, as an alternative to the method shown in FIG. 1, for the recording region to be defined along the long axis measurement slices directly in the third overview measurement data, which has been acquired in the third overview recording Ma3. Then the fourth measurement block Ba4 can be dispensed with completely.

Measurement Block Ba5

Following on from the fourth measurement block Ba4, at a fifth point in time Ta5 during the first heart imaging, a fifth measurement block Ba5 starts. A first diagnostic recording Ma5 is made in the fifth measurement block Ba5, during which first diagnostic measurement data is acquired. The first diagnostic measurement data is also used simultaneously for planning of further measurements in the heart imaging.

The fifth point in time Ta5 lies, in the case shown, 180 s after the start time Ta1 of the first heart imaging. The fifth measurement block Ba4, in the case shown, has a fifth duration of 75 s. Between 2 and 10 seconds, in particular between 4 and 8 seconds, in particular 6 seconds, of the fifth duration are taken up by the pure measurement time of the fifth overview measurement Ma5 for acquiring the first overview measurement data. The pure measurement time of the first diagnostic measurement Ma5 for acquiring the first diagnostic measurement data will typically need between 4 and 8 heartbeats, in particular 6 heartbeats, of the examination object. A remaining duration of the fifth measurement block Ba5 can be taken up partly by a preparation of the acquisition of the first overview measurement data. The remaining duration of the fifth measurement block Ba5 can furthermore be taken up partly by an evaluation or post-processing of the first diagnostic measurement data, in particular in the third evaluation step Ea3.

The first diagnostic recording Ma5 is embodied as a dynamic heart recording along the long axis measurement slices. The first diagnostic recording Ma5 can thus also be referred to as a CINE recording, since a movie loop can be created on the basis of the first diagnostic measurement data, which represents a heart movement during a complete heart cycle. A balanced steady state free precession (bSSFP) magnetic resonance sequence, which is implemented for example as a TrueFISP sequence, is preferably used for acquisition of the first diagnostic measurement data. Basically gradient echo magnetic resonance sequences are well suited for the first diagnostic recording Ma5.

The first diagnostic measurement data is acquired from the recording region (Field of View, FOV) defined in the second evaluation step Ea2 along the long axis measurement slices. The orientation of the slices acquired in the first diagnostic recording Ma5 accordingly corresponds to the orientation of the slices acquired in the fourth overview recording Ma4. However the recording region along the long axis measurement slices in the first diagnostic recording Ma5 is typically optimized by comparison with the recording region of the fourth overview recording Ma4, in particular restricted.

Especially advantageously a maximum of three long axis measurement slices is acquired in the first diagnostic recording Ma5. The long axis measurement slices in this case are in particular not parallel to each other, but are preferably orthogonal to one another. The acquisition of these three long axis measurement slices has proved to be especially suitable, as described in US 2012/0121152 A1: A 4-chamber measurement slice, a 3-chamber measurement slice, a 2-chamber measurement slice. A range of between 1.4 mm and 2 mm, especially preferably 1.7 mm, has proved suitable as pixel resolution within a slice (in-plane resolution). The slice thickness of the long axis measurement slices is preferably selected between 4 mm and 8 mm, especially preferably 6 mm.

The first diagnostic measurement data covers the complete heart cycle, preferably with a temporal resolution of greater than 50 ms. Advantageously the temporal resolution is greater than 35 ms, highly advantageously greater than 25 ms. Higher temporal resolutions are conceivable in this case when suitable acceleration techniques are used. The number of individual images that are acquired during different heart phases depends in this case in particular on the desired temporal resolution. Thus it is conceivable for the first diagnostic measurement data over a heart cycle in a long axis measurement slice to comprise more than 15 individual images, preferably more than 25 individual images, highly advantageously around 50 individual images.

The first diagnostic recording Ma5 can be made when the examination object is holding their breath or when the examination object is breathing freely. If the first diagnostic recording Ma5 is made when the examination object is holding their breath, then typically one breathhold is needed for the acquisition of the first diagnostic measurement data. An acquisition over two breathholds is also conceivable, in particular when an improved time resolution is to be present in the first diagnostic measurement data. In rare cases an acquisition over three or four breathholds is also conceivable.

The first diagnostic measurement data can be acquired segmented over a number of heart cycles of the examination object, using an EKG triggering. It is also conceivable, in particular when a suitable acceleration technique is used, for the first diagnostic measurement data to be recorded in real time.

The parameters of the pixel resolution, the slice thickness and the temporal resolution are advantageously selected such that the first diagnostic measurement data can be fully recorded with the recording sequence used within less than 55 seconds, in particular within less than 50 seconds, advantageously within less than 40 seconds, highly advantageously within less than 35 seconds.

An acceleration technique is employed for acquisition of the first diagnostic measurement data. In particular the use of a compressed sensing acceleration technique is conceivable. The compressed sensing acceleration technique will be explained in greater detail in one of the following sections.

On the basis of the first diagnostic measurement data acquired in the first diagnostic recording Ma5, in a third evaluation step Ea3, an automatic calculation of a position and orientation of short axis measurement slices, which run along the short axis (also referred to as SAX) of the heart, is carried out. These short axis measurement slices can then be acquired in the second diagnostic recording Ma1 in the fifth measurement block Ba5. For more precise information about identifying the short axis measurement slices the reader is again referred to US 2012/0121152 A1.

The automatically established short axis measurement slices are validated by the user in a third user interaction Ia3. There can also be a modification to a number of short axis measurement slices during the third user interaction Ia3. The validation can take place in this case in a way similar to the validation of the long axis measurement slices in the second user interaction Ia2. It is also conceivable, as an alternative to the method shown in FIG. 1, for the short axis measurement slices to be planned on the basis of the fourth overview measurement data with an additional user interaction.

Measurement Block Ba6

Following on from the fifth measurement block Ba5, at a sixth point in time Ta6 during the first heart imaging, there is a sixth measurement block Ba6. In the sixth measurement block Ba6 a fifth overview recording Ma6 is made, during which fifth overview measurement data is acquired.

The sixth point in time Ta6, in the case shown, lies 255 s after the start time Ta1 of the first heart imaging. The sixth measurement block Ba6, in the case shown, has a sixth duration of 45 s. Between 7 and 23 seconds, in particular between 10 and 20 seconds, in particular 15 seconds, of the sixth duration are taken up with the pure measurement time of the fifth overview recording Ma6 for acquiring the fifth overview measurement data. A remaining duration of the sixth measurement block Ba6 can be taken up partly by a preparation of the acquisition of the fifth overview measurement data. The remaining duration of the sixth measurement block Ba6 can furthermore be taken up partly by an evaluation or post-processing of the fifth overview measurement data, in particular in the fourth evaluation step Ea4.

The fifth overview recording Ma6 can be referred to as a short axis localizer or short axis scout. The fifth overview recording Ma6 comprises a measurement of the short axis measurement slices, which has been defined in the third evaluation step Ea3 on the basis of the first diagnostic measurement data and has been validated in the third user interaction Ia3.

The fifth overview recording Ma6 can be made when the examination object is holding their breath or when the examination object is breathing freely. If the fifth overview recording Ma6 is made when the examination object is holding their breath, then typically one breathhold is needed for the acquisition of the fifth overview measurement data.

The number of slices for the fifth overview recording Ma6 and the resolution, indirectly connected thereto the number of items of measurement data recorded, are in these cases typically selected or dimensioned such that the recording of all measurement data that is needed for the fifth overview recording Ma6 can be carried out in one breathhold, i.e. typically within a maximum of 15 seconds.

On the basis of the fifth overview measurement data acquired in the fifth overview recording Ma6, in a fourth evaluation step Ea4 a recording region is defined along the short axis measurement slices. The recording region is in particular restricted to an extent of the heart or a chest cavity of the examination object along the short axis measurement slices. The fourth evaluation step Ea4 can occur in this case in a similar way to the second evaluation step Ea2.

Measurement Block Ba7

Following on from the sixth measurement block Ba6, at a seventh point in time Ta1 during the first heart imaging there is a seventh measurement block Ba7. In the seventh measurement block Ba7 a second diagnostic recording Ma7 is made, during which second diagnostic measurement data is acquired.

The seventh point in time Ta1, in the case shown, lies 300 s after the start time Ta1 of the first heart imaging. The seventh measurement block Ba7, in the case shown, has a seventh duration of 60 s. Between 14 and 30 seconds, in particular between 18 and 26 seconds, in particular 22 seconds, of the seventh duration are taken up with the pure measurement time of the second diagnostic measurement Ma7 for acquiring the second diagnostic measurement data. The pure measurement time of the second diagnostic measurement Ma7 for acquiring the second diagnostic measurement data will typically need between 15 and 25 heartbeats, in particular 20 heartbeats, of the examination object. A remaining duration of the seventh measurement block Ba7 can be taken up partly by a preparation of the acquisition of the second diagnostic measurement data. The remaining duration of the seventh measurement block Ba1 can furthermore be taken up partly by an evaluation or post-processing of the second diagnostic measurement data.

The second diagnostic recording Ma7 is embodied as a dynamic heart recording along the short axis measurement slices. The second diagnostic recording Ma7 can thus also be referred to as a CINE recording, since a movie loop can be created on the basis of the second diagnostic measurement data, which represents a heart movement during a complete heart cycle. A balanced steady state free precession (bSSFP) magnetic resonance sequence, which is implemented for example as a TrueFISP sequence, is preferably used for acquisition of the first diagnostic measurement data. Basically gradient echo magnetic resonance sequences are well suited for the second diagnostic recording Ma7.

The second diagnostic measurement data is acquired from the recording region (Field of View, FOV) defined in the fourth evaluation step Ea4 along the short axis measurement slices. The orientation of the slices acquired in the second diagnostic recording Ma7 accordingly corresponds to the orientation of the slices acquired in the fifth overview recording Ma6. However the recording region along the short axis measurement slices in the second diagnostic recording Ma7 is typically optimized by comparison with the recording region of the fifth overview recording Ma6.

Especially advantageously, in the second diagnostic recording Ma7, a stack consisting of a number of parallel short axis measurement slices is acquired. The number of the acquired short axis measurement slices in this case typically lies between 6 and 14 slices, preferably between 8 and 12 slices. The short axis measurement slices advantageously cover the entire heart from the base of the heart to the tip of the heart. A range of between 1.4 mm and 2 mm, especially preferably 1.7 mm, has proved suitable as pixel resolution within a slice (in-plane resolution). The slice thickness of the short axis measurement slices is preferably selected between 6 mm and 10 mm, especially preferably 8 mm.

The second diagnostic measurement data covers the complete heart cycle, preferably with a temporal resolution of greater than 50 ms. Advantageously the temporal resolution is greater than 35 ms, highly advantageously greater than 25 ms. Higher temporal resolutions are conceivable in this case when suitable acceleration techniques are used. The number of individual images that are acquired during different heart phases, depends in this case in particular on the desired temporal resolution. Thus it is conceivable for the second diagnostic measurement data over a heart cycle in a short axis measurement slice to comprise more than 15 individual images, preferably more than 25 individual images, highly advantageously around 50 individual images.

The second diagnostic recording Ma7 can be made when the examination object is holding their breath or when the examination object is breathing freely. If the second diagnostic recording Ma7 is made when the examination object is holding their breath, then typically two breathholds, in a few cases also only one breathhold, are/is needed for the acquisition of the second diagnostic measurement data. Only occasionally will three or four breathholds be needed.

The second diagnostic measurement data can be acquired segmented over a number of heart cycles of the examination object, using an EKG triggering. It is also conceivable, in particular when a suitable acceleration technique is used, for the second diagnostic measurement data to be recorded in real time.

The parameters of the pixel resolution, the slice thickness and the temporal resolution are advantageously selected such that the second diagnostic measurement data can be fully recorded with the recording sequence used within less than 40 seconds, in particular within less than 35 seconds, advantageously within less than 30 seconds, highly advantageously within less than 25 seconds. An acceleration technique is employed for acquisition of the second diagnostic measurement data. In particular the use of a compressed sensing acceleration technique is once again conceivable.

Fifth Evaluation Step Ea5

Following on from the seventh measurement block Ba7 there is finally a fifth evaluation step Ea5. In this step the first diagnostic measurement data acquired in the first diagnostic recording Ma5 and the second diagnostic measurement data acquired in the second diagnostic recording Ma1 is evaluated.

The evaluation in the fifth evaluation step Ea5 begins in particular after conclusion of the seventh measurement block Ba7, at an eighth point in time Ta8. The eighth point in time Ta8, in the case shown, lies 360 s after the start time Ta1 of the first heart imaging. The eighth point in time Ta8 thus represents an end of the acquisition of the measurement data within the shown first heart imaging. The evaluation in the fifth evaluation step Ea5 lasts 105 s in the case shown and is ended at a ninth point in time Ta9. The ninth point in time Ta9, in the case shown, lies 465 s after the start time Ta1 of the first heart imaging. The ninth point in time Ta9 thus represents an end of the evaluation of the shown first heart imaging.

The evaluation in the fifth evaluation step Ea5 comprises an evaluation of function parameters of a left ventricle of the heart. In the fifth evaluation step Ea5 there can automatically be segmentation of an endocard and/or of an epicard, in particular as a basis for determining the function parameters. The following function parameters can be established automatically or semi-automatically in the fifth evaluation step Ea5, for example with partial user interactions, from the first diagnostic measurement data and the second diagnostic measurement data: A beat volume of the heart, an enddiastolic volume, an endsystolic volume, an ejection fraction, a heart mass. The function parameters can be displayed to the user as a table and/or stored in a database. Reconstructed image data, in particular the movie loops, can continue to be made available to the user on the display unit from the first diagnostic measurement data and second diagnostic measurement data. As an alternative or in addition the image data can also be stored in a database.

FIG. 2—Second Heart Imaging

General Information Relating to the Second Heart Imaging

The second heart imaging, the execution sequence of which is shown in FIG. 2, in particular delivers diagnostic measurement data that can serve as the basis for the evaluation of a heart function of the examination object. In addition the second heart imaging delivers diagnostic measurement data that can serve as a basis for a diagnosis of a possible non ischemic cardiomyopathy of the examination object that might be present. As in the first heart imaging, it is in particular an objective of the second heart imaging in this case, in a second imaging duration that is as short as possible, to record the diagnostic measurement data needed for the assessment of the heart function and diagnosis of a possible non ischemic cardiomyopathy of the examination object that might be present.

The second heart imaging has a second imaging duration, which lasts from a start time Tb1 of the second heart imaging up to a tenth point in time Tb10, at which the recording of measurement data in the second heart imaging is ended. The second imaging duration preferably amounts in this case to a maximum of 18 minutes, advantageously a maximum of 15 minutes, especially advantageously a maximum of 12 minutes, highly advantageously a maximum of 10 minutes. The second imaging duration is in particular embodied as the maximum imaging duration that may not be exceeded in carrying out the second heart imaging. The second imaging duration in this case can include a duration of user interactions or parameter settings for the acquisition of the measurement data. In specific cases it is also conceivable for a period of time for positioning a patient to be calculated into the second imaging duration. As an alternative the second imaging duration can also be characterized in that more than 60 percent, in particular more than 75 percent, highly advantageously more than 90 percent of a series of a number of examinations, which are carried out in accordance with the scheme presented in FIG. 2 for the second heart imaging, adhere to the second imaging duration.

FIG. 2 in this case shows the especially advantageous case in which the second imaging duration of the second heart imaging lasts for 9.5 minutes. After conclusion of the recording of the measurement data in the second heart imaging further time can elapse, in which there is post-processing and/or evaluation of the measurement data.

Description of a Possible Concrete Execution Sequence of the Second Heart Imaging

Preparation of the Second Heart Imaging

The preparation of the second heart imaging can basically comprise a few of or all of the elements that have already been described for the preparation of the first heart imaging. Therefore, as regards the description of the preparation of the second heart imaging, the reader is referred to the description of the preparation of the first heart imaging.

In addition to the preparation of the first heart imaging, in the preparation of the second heart imaging there is an application of contrast medium Cb. In this process a magnetic resonance contrast medium is administered, in particular injected into the examination object. Widely-used magnetic resonance contrast media, such as gadolinium, for example Gd-DTPA, can be used here. The application of contrast medium Cb is advantageously done during the second heart imaging while the examination object is positioned on the patient support facility of the magnetic resonance device for the second heart imaging. The application of contrast medium Cb can also be done during the second heart imaging directly after the positioning of the examination object. Advantageously the application of contrast medium Cb is done during the second heart imaging before the start of the first measurement block Bb1 of the second heart imaging. It is also conceivable for the application of contrast medium Cb to be done directly after the start of the first measurement block Bb1.

Measurement Blocks Bb1-Bb6

The first six measurement blocks Bb1, Bb2, Bb3, Bb4, Bb5, Bb6 of the second heart imaging execute analogously to the first six measurement blocks Ma1, Ma2, Ma3, Ma4, Ma5, Ma6 of the first heart imaging. For the description of these measurement blocks, the reader is therefore referred to the description of the corresponding measurement blocks in the first heart imaging.

The execution sequence of the first six measurement blocks Bb1, Bb2, Bb3, Bb4, Bb5, Bb6 of the second heart imaging will be briefly summarized once again at this point, wherein, as regards more comprehensive descriptions and alternative execution options, the reader is referred to the description of the first six measurement blocks Ma1, Ma2, Ma3, Ma4, Ma5, Ma6 in FIG. 1:

A first overview recording Mb1 is made in the first measurement block Bb1 of the second heart imaging. On the basis of the first overview measurement data acquired in the first overview recording Mb1, the heart of the examination object is positioned by way of a first user interaction Ib1 in the isocenter of the magnetic resonance device.

A second overview recording Mb2 is made in the second measurement block Bb2, in which the heart is positioned in the isocenter of the magnetic resonance device.

A third overview recording Mb3 is made in the third measurement block Bb3. On the basis of the third overview measurement data acquired in the third overview recording Mb3, in a first evaluation step Eb1, an orientation of long axis measurement slices can be established. The automatically established long axis measurement slices are validated by the user in a second user interaction Ib2.

A fourth overview recording Mb4 is made in the fourth measurement block Bb4, wherein on the basis of the fourth overview measurement data acquired here, in a second evaluation step Eb2 a recording region is defined along the long axis measurement slices.

From this recording region, in the fifth measurement block Bb5, in a first diagnostic recording Mb5, first diagnostic measurement data is acquired dynamically in the sense of a CINE recording along the long axis of the heart. On the basis of the first diagnostic measurement data, in a third evaluation step Eb3, an automatic calculation of a position and orientation of short axis measurement slices is carried out. The automatically established short axis measurement slices are validated by the user in a third user interaction Ib3.

Thus, in a fifth overview recording Mb6, in the sixth measurement block Bb6, fifth overview measurement data can be acquired from the short axis measurement slices. On the basis of the fifth overview measurement data acquired in the fifth overview recording Mb6, in a fourth evaluation step Eb4, a recording region is defined along the short axis measurement slices.

Measurement Block Bb7

Following on from the sixth measurement block Bb6, a seventh measurement block Bb7 starts at a seventh point in time Tb7 during the second heart imaging. A second diagnostic recording Mb7 is made in the seventh measurement block Bb7, during which second diagnostic measurement data is acquired.

The seventh point in time Tb7, in the case shown, lies 300 s after the start time Tb1 of the second heart imaging. The seventh measurement block Bb7, in the case shown, has a seventh duration of 120 s. Between 21 and 45 seconds, in particular between 27 and 39 seconds, in particular 33 seconds, of the seventh duration are taken up with the pure measurement time of the second diagnostic measurement Mb7 for acquiring the second diagnostic measurement data. The pure measurement time of the second diagnostic measurement Mb7 for acquiring the second diagnostic measurement data will typically need between 22 and 38 heartbeats, in particular 30 heartbeats, of the examination object. A remaining duration of the seventh measurement block Bb7 can be taken up partly by a preparation of the acquisition of the second diagnostic measurement data. The remaining duration of the seventh measurement block Bb7 can furthermore be taken up partly by an evaluation or post-processing of the second diagnostic measurement data.

The second diagnostic recording Mb7 is embodied as a T1-mapping measurement. This means that during the second diagnostic recording Mb7 a spatially-resolved distribution of a T1 relaxation time (also called a T1 map) in the heart of the examination object is quantified. The acquired T1 map can be reconstructed directly after the conclusion of the second diagnostic recording Mb7 and be provided for diagnosis. Different methods for acquiring the T1 map are known to the person skilled in the art, so that the methods will not be discussed in any greater detail here.

The second diagnostic measurement data is acquired from the recording region (Field of View, FOV) along the short axis measurement slice defined in the fourth evaluation step Eb4. The orientation of the slices acquired in the second diagnostic recording Mb7 accordingly corresponds to the orientation of the slices acquired in the fifth overview recording Mb6. However the recording region along the short axis measurement slices in the second diagnostic recording Mb7 is typically restricted compared to the recording region of the fifth overview recording Mb6.

Especially advantageously a stack consisting of a number of parallel short axis measurement slices is acquired in the second diagnostic recording Mb7. The number of the acquired short axis measurement slices typically lies in this case between 1 and 5 slices, preferably between 2 and 4 slices. The short axis measurement slices, for which the T1 relaxation times are measured, are in particular arranged such that, if possible, they cover the left ventricle of the heart, advantageously a central region of the left ventricle.

The second diagnostic recording Mb7 can be made when the examination object is holding their breath or when the examination object is breathing freely. If the second diagnostic recording Mb7 is made when the examination object is holding their breath, then typically three breathholds, in a few cases more than three breathholds, are needed for the acquisition of the second diagnostic measurement data. Only occasionally will fewer than three breathholds be needed.

There can be a user interaction in the seventh measurement block Mb7, in which the measurement region is set along the short axis measurement slices for the second diagnostic recording Mb7 and/or the third diagnostic recording Mb8 and/or the fourth diagnostic recording Mb9. Different measurement regions can be defined here along the short axis measurement slices or different slice stacks for the different diagnostic recordings Mb7, Mb8, M9.

Measurement Block Bb8

Following on from the seventh measurement block Bb1, at an eighth point in time Tb8, an eighth measurement block Bb8 starts during the second heart imaging. A third diagnostic recording Mb8 is made in the eighth measurement block Bb8, during which third diagnostic measurement data is acquired.

The eighth point in time Tb8, in the case shown, lies 420 s after the start time Tb1 of the second heart imaging. The eighth measurement block Bb8, in the case shown, has an eighth duration of 120 s. Between 21 and 45 seconds, in particular between 27 and 39 seconds, in particular 33 seconds, of the eighth duration are taken up with the pure measurement time of the third diagnostic measurement Mb8 for acquiring the third diagnostic measurement data. The pure measurement time of the third diagnostic measurement Mb8 for acquiring the third diagnostic measurement data will typically need at least 20 heartbeats, in particular at least 26 heartbeats, of the examination object. A remaining duration of the eighth measurement block Bb8 can be taken up partly by a preparation of the acquisition of the third diagnostic measurement data. The remaining duration of the eighth measurement block Bb8 can furthermore be taken up partly by an evaluation or post-processing of the third diagnostic measurement data.

The third diagnostic recording Mb8 is embodied as a delayed enhancement measurement, also called a late enhancement measurement. In this way, in the eighth diagnostic recording Mb8, an accumulation of the contrast medium in a heart structure, for example in the myocardium and/or pericardium administered during the application of contrast medium Cb to the examination object, is measured. Image data reconstructed from the third diagnostic measurement data can be reconstructed directly after the conclusion of the third diagnostic recording Mb8 and provided for the diagnosis.

A gradient echo sequence, in particular a gradient echo sequence in the stationary state, such as for example a balanced steady state free precession (bSSFP) magnetic resonance sequence, can be employed advantageously for the third diagnostic recording Mb8. For optimization of a contrast the third diagnostic recording Mb8 can use a saturation of tissue signals, for example by way of an inversion pulse or by using a Phase Sensitive Inversion Recovery (PSIR) technique.

The third diagnostic measurement data is acquired both along the long axis measurement slices and also along the short axis measurement slices. This enables the first diagnostic measurement data to be acquired both from the recording region along the long axis measurement slices defined in the second evaluation step Eb2 and also from the recording region along the short axis measurement slices defined in the fourth evaluation step Eb4. In this case the eighth measurement block Bb8 can comprise a user interaction not shown in FIG. 2, in which the recording region for the delayed enhancement measurement, in particular the long axis measurement slices and/or short axis measurement slices to be recorded, can be validated and/or modified. The complete/part acceptance of the recording regions defined in the second evaluation step and/or in the fourth evaluation step also enables the user interaction to be dispensed with however.

The third diagnostic recording Mb8 can be made when the examination object is holding their breath or when the examination object is breathing freely. If the third diagnostic recording Mb8 is made when the examination object is holding their breath, then typically five breathholds, in a few cases more than five breathholds, will be needed for the acquisition of the third diagnostic measurement data. Occasionally fewer than five breathholds will be needed.

Measurement Block Bb9

Following on from the eighth measurement block Bb8, at a ninth point in time Tb9 during the second heart imaging, a ninth measurement block Bb9 starts. A fourth diagnostic recording Mb9 is made in the ninth measurement block Bb9, during which fourth diagnostic measurement data is acquired. The ninth point in time Tb9, in the case shown, lies 540 s after the start time Tb1 of the second heart imaging. The ninth measurement block Bb9, in the case shown, has a ninth duration of 30 s.

The ninth measurement block Bb9 of the second heart imaging is embodied in a similar way to the seventh measurement block Ba7 of the first heart imaging. Thus, for the description of the ninth measurement block Bb9, in particular of the fourth diagnostic recording Mb9, of the second heart imaging, the reader is referred to the description for the seventh measurement block Ba7, in particular of the second diagnostic recording Ma1, of the first heart imaging.

The fourth diagnostic recording Mb9 is thus again embodied as a dynamic heart recording along the short axis measurement slices. The short axis measurement slices can if necessary once again be modified and/or validated in a user interaction in the ninth measurement block Bb9 not shown in FIG. 2.

Fifth Evaluation Step Eb5

Following on from the ninth measurement block Bb9, there is finally a fifth evaluation step Eb5. In this step the first diagnostic measurement data acquired in the first diagnostic recording Mb5 and the fourth diagnostic measurement data acquired in the fourth diagnostic recording Mb9 is evaluated. In addition, in the fifth evaluation step Eb5, there can be evaluations of diagnostic measurement data acquired in the seventh measurement block Bb7 and/or in the eighth measurement block Bb8.

The evaluation in the fifth evaluation step Eb5 begins in particular after the conclusion of the ninth measurement block Bb9 at a tenth point in time Tb10. The tenth point in time Tb10, in the case shown, lies 570 s after the start time Tb1 of the second heart imaging. The tenth point in time Tb10 thus represents an end of the acquisition of the measurement data within the shown second heart imaging. The evaluation in the fifth evaluation step Eb5 lasts for 90 s in the case shown and is ended at an eleventh point in time Tb11. The eleventh point in time Tb11, in the case shown, lies 660 s after the start time Tb1 of the second heart imaging. The eleventh point in time Tb11 thus represents an end of the evaluation of the second heart imaging shown.

The evaluation of the function parameters in the fifth evaluation step Eb5 of the second heart imaging based on the first diagnostic measurement data and fourth diagnostic measurement data is done in a similar way to the fifth evaluation step Ea5 of the first heart imaging. Therefore the reader is referred at this point to the description of the fifth evaluation step Ea5 of the first heart imaging.

Provided this has not already happened in the seventh measurement block Bb7, in the fifth evaluation step Eb5 there can additionally be a calculation and/or a provision of the T1 map based on the second diagnostic measurement data. Furthermore, if this has not already been done in the eighth measurement block Bb8, the third diagnostic measurement data from the delayed enhancement measurement can also be evaluated in the fifth evaluation step Eb5.

FIG. 3—Third Heart Imaging

General Information Relating to the Third Heart Imaging

The third heart imaging, the execution sequence of which is shown in FIG. 3, in particular delivers diagnostic measurement data that can serves as the basis for an assessment of a heart function of the examination object. In addition the third heart imaging delivers diagnostic measurement data that can serve as a basis for a diagnosis of the possible presence of a non ischemic cardiomyopathy of the examination object. In addition the third heart imaging delivers diagnostic measurement data that can serve as a basis for a diagnosis of the possible presence of an ischemic cardiomyopathy of the examination object. As with the first heart imaging and the second heart imaging, it is in particular an objective of the third heart imaging in this case to record, in a third imaging duration that is as short as possible, the diagnostic measurement data needed for the assessment of the heart function and for diagnosis of the possible presence of a non ischemic cardiomyopathy or ischemic cardiomyopathy of the examination object.

The third heart imaging has a third imaging duration, which lasts from a start time Tc1 of the third heart imaging up to a thirteenth point in time Tc13, at which the recording of measurement data in the third heart imaging is ended. The third imaging duration preferably amounts in this case to a maximum of 22 minutes, advantageously a maximum of 19 minutes, especially advantageously a maximum of 17 minutes, highly advantageously a maximum of 15 minutes. The third imaging duration is in particular embodied as the maximum imaging duration that may not be exceed when carrying out the third heart imaging. The third imaging duration in this case can include a period of time of user interactions or parameter settings for the acquisition of the measurement data. In specific cases it is also conceivable for a period of time for positioning a patient to be calculated into the third imaging duration. As an alternative the third imaging duration can also be characterized in that more than 60 percent, in particular more than 75 percent, highly advantageously more than 90 percent of a series of a number of examinations, which are carried out in accordance with the scheme presented in FIG. 3 for the third heart imaging, adhere to the third imaging duration.

In FIG. 3 in this case the especially advantageous case is shown in which the third imaging duration of the third heart imaging lasts 15 minutes. After conclusion of the recording of the measurement data in the third heart imaging yet further time can elapse, in which there is a post-processing and/or evaluation of the measurement data.

Description of a Possible Concrete Execution Sequence of the Third Heart Imaging

Preparation of the Third Heart Imaging

The preparation of the third heart imaging can basically comprise a few of or all of the elements that have already been described for the preparation of the first heart imaging. Therefore, as regards the description of the preparation of the third heart imaging, the reader is referred to the description of the preparation of the first heart imaging.

The contrast medium Cc for the third heart imaging is applied, by contrast with the application of contrast medium Cb for the second heart imaging, not during the preparation of the third heart imaging, but during the measuring sequence of the third heart imaging. In the case shown in FIG. 3 the application of contrast medium Cc is done immediately before the beginning of the seventh measurement block Bc7 of the third heart imaging.

Measurement Blocks Bc1-Bc6

The first six measurement blocks Bc1, Bc2, Bc3, Bc4, Bc5, Bc6 of the third heart imaging execute analogously to the first six measurement blocks Ma1, Ma2, Ma3, Ma4, Ma5, Ma6 of the first heart imaging. For the description of these measurement blocks, the reader is therefore referred to the description of the corresponding measurement blocks in the first heart imaging.

The execution sequence of the first six measurement blocks Bc1, Bc2, Bc3, Bc4, Bc5, Bc6 of the third heart imaging will be briefly summarized once again at this point, wherein, as regards more comprehensive descriptions and alternative execution options, the reader is referred to the description of the first six measurement blocks Ma1, Ma2, Ma3, Ma4, Ma5, Ma6 in FIG. 1:

A first overview recording Mc1 is made in the first measurement block Bc1 of the third heart imaging. On the basis of the first overview measurement data acquired in the first overview recording Mc1, the heart of the examination object is positioned by way of a first user interaction Ic1 in the isocenter of the magnetic resonance device.

A second overview recording Mc2 is made in the second measurement block Bc2, in which the heart is positioned in the isocenter of the magnetic resonance device.

A third overview recording Mc3 is made in the third measurement block Bc3. On the basis of the third overview measurement data acquired in the third overview recording Mc3, in a first evaluation step Ec1, an orientation of long axis measurement slices can be established. The automatically established long axis measurement slices are validated in a second user interaction Ic2 by the user.

A fourth overview recording Mc4 is made in the fourth measurement block Bc4, wherein, on the basis of the fourth overview measurement data acquired here, in a second evaluation step Ec2, a recording region is defined along the long axis measurement slices.

From this recording region, in the fifth measurement block Bc5, in a first diagnostic recording Mc5, first diagnostic measurement data is acquired dynamically in the sense of a CINE recording along the long axis of the heart. On the basis of the first diagnostic measurement data, in a third evaluation step Ec3, an automatic calculation of a position and orientation of short axis measurement slices is carried out. The automatically established short axis measurement slices are validated by the user in a third user interaction Ic3.

Thus, in a fifth overview recording Mc6, in the sixth measurement block Bc6, fifth overview measurement data can be acquired from the short axis measurement slices. On the basis of the fifth overview measurement data acquired in the fifth overview recording Mc6, in a fourth evaluation step Ec4 a recording region is defined along the short axis measurement slices.

Measurement Block Bc7

Following on from the sixth measurement block Bc6, a seventh measurement block Bc7 starts at a seventh point in time Tc7 during the second heart imaging. A second diagnostic recording Mc7 is made in the seventh measurement block Bc7, during which second diagnostic measurement data is acquired.

The seventh point in time Tc7, in the case shown, lies 300 s after the start time Tc1 of the third heart imaging. The seventh measurement block Bc7, in the case shown, has a seventh duration of 60 s. Between 4 and 14 seconds, in particular between 7 and 12 seconds, of the seventh duration are taken up with the pure measurement time of the sixth overview measurement Mb7 for acquiring the sixth overview measurement data. The pure measurement time of the sixth overview measurement Mb7 for acquiring of the sixth overview measurement data will typically need between 3 and 12 heartbeats, in particular between 5 and 10 heartbeats, of the examination object. A remaining duration of the seventh measurement block Bb7 can be taken up partly by a preparation of the acquisition of the sixth overview measurement data. The remaining duration of the seventh measurement block Bb7 can furthermore be taken up partly by an evaluation or post-processing of the sixth overview measurement data.

The sixth overview recording Mc7 is embodied as a test perfusion measurement. In the test perfusion measurement there is in particular not yet any influence of a stress medicament on the examination object. Despite this the stress medicament can already be administered during the seventh measurement block Bc7 to the examination object, so that the effect of the stress medicament occurs a few minutes later during the carrying out of the eighth measurement block Bc8. Furthermore the test perfusion measurement is made without prior application of a contrast medium. The test perfusion measurement is carried out in particular for the reason of verifying recording parameters for the subsequent stress perfusion measurement in the eighth measurement block Bc8. In this way a repetition of the subsequent stress perfusion measurement can advantageously be avoided. A repetition of the subsequent stress perfusion measurement would be especially disadvantageous because of the application of contrast medium Cc or the administration of the stress medicament.

The test perfusion measurement can in particular be carried out with the same recording parameters as the stress perfusion measurement in the eighth measurement block Bc8, so that for the description of the recording parameters the reader is referred to the description of the eighth measurement block Bc8. Options for carrying out the test perfusion measurement are known to the person skilled in the art in this case, so that the options will not be discussed in any greater detail here.

Measurement Block Bc8

Following on from the seventh measurement block Bc7 there is an eighth measurement block Bc8. A second diagnostic recording Mc8 is made in the eighth measurement block Bc8, during which second diagnostic measurement data is acquired.

The eighth point in time Tc8, in the case shown, lies 360 s after the start time Tc1 of the third heart imaging. The eighth measurement block Bc8, in the case shown, has an eighth duration of 150 s. Between 32 and 96 seconds, in particular between 56 and 72 seconds, of the eighth duration are taken up by the pure measurement time of the second diagnostic measurement Mb8 for acquiring the second diagnostic measurement data. The pure measurement time of the second diagnostic measurement Mb8 for acquiring the second diagnostic measurement data will typically need between 24 and 78 heartbeats, in particular between 40 and 60 heartbeats, of the examination object. A remaining duration of the eighth measurement block Bb8 can be taken up partly by a preparation of the acquisition of the second diagnostic measurement data. The remaining duration of the eighth measurement block Bb8 can furthermore be taken up partly by an evaluation or post-processing of the second diagnostic measurement data.

At the beginning of the eighth measurement block Mc8 in particular a stress medicament, for example adenosine or dipyridamole, is administered to the examination object. As already described, the stress medicament can also already be administered to the examination object during the seventh measurement block Bc7, so that the effect of the stress medicament sets in a few minutes later during the second diagnostic recording Mc8. Furthermore, at the beginning of the eighth measurement block Mc8, the application of contrast medium Cc for the third heart imaging occurs.

The second diagnostic recording Mc8 is embodied as a stress perfusion measurement. In the stress perfusion measurement in particular a perfusion of the contrast medium administered to the examination object through blood vessels can be measured. It is also conceivable for the second diagnostic recording Mc8 to be embodied as a perfusion measurement without prior application of the stress medicament. Options for perfusion measurement of the heart are known to the person skilled in the art in this case, so that the options will not be discussed in any greater detail here.

The same recording parameters can be used for the second diagnostic recording Mc8 as for the sixth overview measurement Mc7. The difference between the sixth overview measurement Mc7 and the second diagnostic recording Mc8 lies in particular in the modified stress of the heart of the examination object by the application of the stress medicament or the application of contrast medium Cc, as well as a longer acquisition time, in order to enable the contrast medium spread to be observed.

A gradient echo sequence, preferably a balanced steady state free precession (bSSFP) magnetic resonance sequence or a gradient echo sequence with an accelerated readout of the signals (TurboFLASH magnetic resonance sequence) can be used for the second diagnostic recording Mc8. Use of echo planar imaging (an EPI magnetic resonance sequence) is also conceivable.

The second diagnostic measurement data can be provided and/or evaluated directly after its acquisition. For example perfusion parameters, such as for example a speed of a contrast medium accumulation (perfusion up-slope), can be quantified and provided directly after the conclusion of the second diagnostic measurement Mc8.

The second diagnostic measurement data is acquired from the recording region (Field of View, FOV) along the short axis measurement slices defined in the fourth evaluation step Ec4. In particular the second diagnostic measurement data and the sixth overview measurement data is acquired from the same recording region. The orientation of the slices acquired in the second diagnostic recording Mc8 accordingly corresponds to the orientation of the slices acquired in the fifth overview recording Mc6. However the recording region along the short axis measurement slices in the second diagnostic recording Mc8 is typically restricted when compared to the recording region of the fifth overview recording Mc6.

Especially advantageously, in the second diagnostic recording Ma8, a stack consisting of a number of parallel short axis measurement slices is acquired. The number of the acquired short axis measurement slices in this case typically lies between 1 and 5 slices, preferably between 2 and 4 slices. The short axis measurement slices are in particular positioned in the middle of the heart. The positioning and/or selection of the short axis measurement slices to be recorded in the second diagnostic recording Mc8 can be undertaken in a user interaction not shown in FIG. 3. It is conceivable, as well as the number of parallel short axis measurement slices in the second diagnostic recording Mc8, additionally to acquire a measurement slice along a long axis measurement slice.

The second diagnostic recording Mc8 can be made when the examination object is holding their breath or when the examination object is breathing freely. If the second diagnostic recording Mc8 is made when the examination object is holding their breath, then typically one breathhold is needed for the acquisition of the second diagnostic measurement data, in order be able advantageously to measure the perfusion up-slope.

Measurement Block Bc9

Following on from the eighth measurement block Bc8, at a ninth point in time Tc9 during the third heart imaging, a ninth measurement block Bc9 starts. A third diagnostic recording Mc9 is made in the ninth measurement block Bc9, during which third diagnostic measurement data is acquired.

The ninth point in time Tc9, in the case shown, lies 510 s after the start time Tc1 of the third heart imaging. The ninth measurement block Bc9, in the case shown, has an ninth duration of 30 s. Between 5 and 15 seconds, in particular between 8 and 12 seconds, of the ninth duration are taken up by the pure measurement time of the third diagnostic recording Mc9 for acquiring the third diagnostic measurement data. A remaining duration of the ninth measurement block Bc9 can be taken up partly by preparation of the acquisition of the third diagnostic measurement data. The remaining duration of the ninth measurement block Bc9 can furthermore be taken up partly by an evaluation or post-processing of the third diagnostic measurement data.

The third diagnostic recording Mc9 is embodied as a thorax recording. In the thorax recording the third diagnostic measurement data is acquired from a thorax region of the examination object. A spin echo sequence, in particular a turbo spin echo sequence, for example a Half-Fourier Acquisition Single-Shot Turbo Spin Echo magnetic resonance sequence (HASTE magnetic resonance sequence), can be used for the third diagnostic recording Mc9. As an alternative, a balanced steady state free precession (bSSFP) magnetic resonance sequence can also be used for the third diagnostic recording Mc9. Measurement slices in coronal and/or transversal orientation in relation to the examination object can advantageously be acquired for the thorax recording.

In particular the sequence of the ninth measurement block Mc9 and of the tenth measurement block Mc10 can be swapped over as required. The tenth measurement block Mc10 begins in this case at the ninth point in time Tc9 of the third heart imaging.

It is conceivable for the ninth measurement block Bc9 additionally to be inserted into the first heart imaging in accordance with FIG. 1 or into the second heart imaging in accordance with FIG. 2. This leads in particular to a lengthening of the imaging durations of these heart imagings.

Measurement Block Bc10

Following on from the ninth measurement block Bc9, a tenth measurement block Bc10 starts at a tenth point in time Tc10 during the third heart imaging. A fourth diagnostic recording Mc10 is made in the tenth measurement block Bc10, during which fourth diagnostic measurement data is acquired. The tenth point in time Tc10, in the case shown, lies 540 s after the start time Tc1 of the third heart imaging. The tenth measurement block Bc10, in the case shown, has a tenth duration of 120 s.

The tenth measurement block Bc10 of the third heart imaging is embodied analogously to the seventh measurement block Bb7 of the second heart imaging. Thus, for the description of the tenth measurement block Bb10, in particular of the fourth diagnostic recording Mb10 of the third heart imaging, the reader is referred to the description of the seventh measurement block Bb7, in particular of the second diagnostic recording Mb7 of the second heart imaging.

The fourth diagnostic recording Mc10 is thus again embodied as a T1 mapping. The short axis measurement slices can if necessary be modified and/or validated again in a user interaction in the tenth measurement block Bc10 not shown in FIG. 3.

In particular the sequence of the ninth measurement block Mc9 and of the tenth measurement block Mc10 can be swapped over as required. The tenth measurement block Mc10 begins in this case at the ninth point in time Tc9 of the third heart imaging. It is also conceivable for the tenth measurement block Bc10, i.e. the T1 mapping measurement, to occur before the application of contrast medium Cc, wherein a lengthening of the third imaging duration must be taken into account.

Measurement Block Bc11

Following on from the tenth measurement block Bc10, an eleventh measurement block Bc11 starts at an eleventh point in time Tc11 during the third heart imaging. A fifth diagnostic recording Mc11 is made in the eleventh measurement block Bc11, during which fifth diagnostic measurement data is acquired. The eleventh point in time Tc11, in the case shown, lies 660 s after the start time Tc1 of the third heart imaging. The eleventh measurement block Bc11, in the case shown, has an eleventh duration of 60 s.

The eleventh measurement block Bc11 of the third heart imaging is embodied analogously to the seventh measurement block Ba7 of the first heart imaging. Thus, for the description of the eleventh measurement block Bb11, in particular of the fifth diagnostic recording Mb11 of the third heart imaging, the reader is referred to the description of the seventh measurement block Ba7, in particular of the second diagnostic recording Ma1 of the first heart imaging.

The fifth diagnostic recording Mc11 is thus again embodied as a dynamic heart recording along the short axis measurement slices. The short axis measurement slices can if necessary be modified and/or validated again in a user interaction in the eleventh measurement block Bc11 not shown in FIG. 3.

Measurement Block Bc12

Following on from the eleventh measurement block Bc11 at a twelfth point in time Tc12, a twelfth measurement block Bc12 starts during the third heart imaging. A sixth diagnostic recording Mc12 is made in the twelfth measurement block Bc12, during which sixth diagnostic measurement data is acquired. The twelfth point in time Tc12, in the case shown, lies 720 s after the start time Tc1 of the third heart imaging. The twelfth measurement block Bc12, in the case shown, has a twelfth duration of 180 s.

The twelfth measurement block Bc12 of the third heart imaging is embodied analogously to the eighth measurement block Bb8 of the second heart imaging. Thus, for the description of the twelfth measurement block Bc12, in particular of the sixth diagnostic recording Mc12 of the third heart imaging, the reader is referred to the description of the eighth measurement block Bb8, in particular of the third diagnostic recording Mb7 of the second heart imaging.

The sixth diagnostic recording Mc12 is thus again embodied as a delayed enhancement measurement along the short axis measurement slices and the long axis measurement slices. The short axis measurement slices and/or long axis measurement slices can if necessary be modified and/or validated again in a user interaction in the twelfth measurement block Bc12 not shown in FIG. 3.

Fifth Evaluation Step Ec5

Following on from the twelfth measurement block Bc12 there is finally a fifth evaluation step Ec5. In this step the first diagnostic measurement data acquired in the first diagnostic recording Mc5 and the fifth diagnostic measurement data acquired in the fifth diagnostic recording Mc11 are evaluated. In addition, in the fifth evaluation step Ec5, there can be evaluations of the diagnostic measurement data acquired in the further measurement blocks Mc8, Mc9, Mc10, Mc12.

The evaluation in the fifth evaluation step Ec5 begins in particular after conclusion of the twelfth measurement block Bc12 at a thirteenth point in time Tc13. The thirteenth point in time Tc13, in the case shown, lies 900 s after the start time Tc1 of the third heart imaging. The thirteenth point in time Tc13 thus represents an end of the acquisition of the measurement data within the third heart imaging shown. The evaluation in the fifth evaluation step Ec5 lasts, in the case shown, for 60 s and is ended at a fourteenth point in time Tc14. The fourteenth point in time Tc14, in the case shown, lies 960 s after the start time Tc1 of the third heart imaging. The fourteenth point in time Tc14 thus represents an end of the evaluation of the measurement data within the third heart imaging shown.

The function parameters in the fifth evaluation step Ec5 of the third heart imaging based on the first diagnostic measurement data and fifth diagnostic measurement data are evaluated analogously to the fifth evaluation step Ea5 of the first heart imaging. Therefore the reader is referred at this point to the description of the fifth evaluation step Ea5 of the first heart imaging.

Once again, in the fifth evaluation step Ec5, there can be an evaluation of the T1 mapping measurement and the delayed enhancement measurement. In addition, in the fifth evaluation step Ec5, provided this has not yet been done in the eighth measurement block Bc8, the perfusion measurement data acquired in the second diagnostic recording Mc8 is evaluated. Finally an evaluation of the third diagnostic measurement data acquired in the third diagnostic recording Mc9 is also conceivable in the fifth evaluation step Ec5, provided this has not yet been done in the ninth measurement block Bc9.

Description of Acceleration and Automation Techniques

In order to be able to record informative diagnostic measurement data within the maximum predetermined imaging duration, different acceleration techniques and/or automation techniques are used in the execution sequences presented for heart imaging. A few of the acceleration techniques and automation techniques used in heart imaging will be presented below. In such cases the techniques presented can be used individually, but can also be combined. A few of the techniques presented are applicable both to the first heart imaging, the second heart imaging and the third heart imaging. Where indicated, techniques can also be presented in this section that are only applicable to one of the three execution sequences presented for heart imaging.

Reduction of User Interactions

During a heart imaging shown in FIG. 1-FIG. 3 there are a maximum of five user interactions. Especially advantageously the number of user interactions during an overall heart imaging is restricted to four. Highly advantageously only the three user interactions shown occur for each heart imaging. In addition, before the start of the heart imaging, there can be a user interaction for registration of the examination object and/or for entering the patient-specific features. The combined number of overview recordings and diagnostic recordings during the heart imaging is in particular larger, especially advantageously at least twice as large, as the number of user interactions during the heart imaging.

Between the first diagnostic recording in the heart imaging and the second diagnostic recording, in the case shown in FIG. 1-FIG. 3, there is one user interaction. In particular more, in particular twice as many, user interactions occur before the beginning of the first diagnostic recording than there are user interactions between the first diagnostic recording and the second diagnostic recording. Furthermore advantageously at least an equal number, highly advantageously more, automatic evaluation steps than user interactions occur during the heart imaging.

The number of user interactions is advantageously reduced by suitable automation measures in the heart imaging. The third overview recording in particular is to be highlighted here. The third overview measurement data acquired here will be used for automatic positioning of the long axis measurement slices. The short axis measurement slices can then be defined automatically on the basis of the first diagnostic measurement data. Measurement parameters, such as for example slice positionings and/or shim volumes, can be automatically copied between different measurement blocks. Automatic voice commands can also be output to the examination object, so that the user does not have to concentrate on these while the heart imaging is being carried out.

At the same time the protocol used for the heart imaging can be dynamically adapted to patient-specific features. Thus a recording region for the diagnostic measurement data can be defined automatically on the basis of a size of the patient. It is also conceivable for the acquisition of the measurement data to be done automatically during a regular or steady heartbeat of the examination object. Furthermore it is advantageous to adapt the durations of the measurements to a maximum breathhold of the examination object. The maximum breathhold can be entered manually into the system as a patient-specific feature for example before the beginning of the measurement by the user, by selecting it from a list of suggestions for example.

At the same time it is advantageous, at the points in the heart imaging at which a user interaction is needed, for the user to be given instructions for the respective user interaction, advantageously directly on the display unit. Advantageously the user will already be provided with suggestions, which he then simply has to accept or modify. At the same time, for a user interaction needed, suitable tools for carrying out the user interaction are advantageously displayed directly to the user. In this way the user can be guided through the workflow during the heart imaging. The instructions to the user for the user interaction enable a time needed for the user interaction to be reduced. A usual time for the user interaction can in this way amount to a maximum of half a minute, advantageously a maximum of 20 seconds, especially advantageously a maximum of 10 seconds, highly advantageously a maximum of 5 seconds.

Overall the intelligently placed user interactions, which advantageously only take place at defined points in time in the execution sequence of the heart imaging, make it possible, by comparison with conventional heart examinations, to speed up the execution sequence of the heart imaging such that the acquisition of the diagnostic measurement data needed for assessing the heart, for example the heart function, of the examination object, will be made possible within the maximum imaging duration.

The evaluation of the first diagnostic measurement data and second diagnostic measurement data, in particular in the last evaluation step, especially advantageously takes place automatically. The image data created in this case can automatically be provided with informative designations, so that it can be found again especially easily by the doctor making the diagnosis. Thus for example can be quantified automatically, in the sense of an “inline-processing” directly after the measurement. For example the perfusion measurement data acquired in the third heart imaging can also be quantified directly in the sense of the “inline-processing”.

The reduction of the number of user interactions needed can lead to a shorter imaging duration needed for the heart imaging. Also this makes the heart imaging especially user-friendly to operate. The results of the heart imaging can be especially robust, since they are less susceptible to user errors. The intelligent placing of the user interactions in the execution sequence of the heart imaging can thus improve the technical safety of the execution sequence of the heart imaging. At the same time standardized diagnostic measurement data can be acquired in the heart imaging in this way. Also an imaging duration for the heart imaging is standardized because of the automations and can thus be well predicted. This can lead to an improved planning of a loading of the magnetic resonance device.

General Arrangement of Overview Recordings and Diagnostic Recordings

In particular there are a maximum of six, in most cases five, overview recordings in the heart imaging. Through further automations already described it can be possible to combine the first overview recording and the second overview recording with each other here, which enables further measurement time to be saved. While the third overview recording will be present in most cases, it is also conceivable, in specific cases, to do without the fourth overview recording and/or the fifth overview recording. The recording region for the acquisition of the diagnostic measurement data along the long axis of the heart and of the diagnostic measurement data along the short axis of the heart can in this case be defined directly based on the third overview measurement data acquired in the third overview recording.

During the heart imaging an overview recording is made between the first diagnostic recording and the second diagnostic recording in the cases shown. In this way the overview recordings and the diagnostic recordings are carried out at least partly nested in one another in their temporal execution sequence. In particular there are more, in particular more than twice as many, overview recordings before the first diagnostic recording as there are overview recordings between the first diagnostic recording and the second diagnostic recording.

Overall the intelligent, in particular nested, arrangement by comparison with conventional heart examinations of the measurement blocks for the overview recordings and diagnostic recordings, in particular in combination with the harmonization with one another of their duration in time, makes it possible to speed up the execution sequence of the heart imaging such that the acquisition of the diagnostic measurement data needed for the assessment of the heart, for example the heart function, of the examination object is made possible within the maximum imaging duration.

Specifically in the first heart imaging the relevant diagnostic information for the assessment of the heart function can be acquired in two diagnostic recordings Ma5, Ma7. In this way the number of overview recordings Ma1, Ma2, Ma3, Ma4, Ma6 in the first heart imaging is in particular more than twice as large as the number of diagnostic recordings Ma5, Ma7. As an alternative it is also conceivable for the number of overview recordings Ma1, Ma2, Ma3, Ma4, Ma6 in the first heart imaging to be precisely twice as large as the number of diagnostic recordings Ma5, Ma7.

Temporal Arrangement of the Diagnostic Recordings in Relation to Application of Contrast Medium

Specifically in the second heart imaging and the third heart imaging there is at least one application of contrast medium Cb, Cc in each case. The first heart imaging can be carried out without application of contrast medium. The application of contrast medium Cb, Cc is arranged in time here such that, for the following diagnostic recordings, there is a most suitable possible accumulation of the administered contrast medium in the heart tissue of the examination object. At the same time the diagnostic recordings in the second heart imaging and the third heart imaging following the application of contrast medium Cb, Cc are arranged in time especially advantageously in relation to the accumulation of the administered contrast medium.

Advantageously the application of contrast medium Cb is done in the second heart imaging before the start of the first measurement block Bb1 of the second heart imaging. The eighth point in time Tb8 is selected in this case such that at least 8 minutes, in particular at least 9 minutes, advantageously at least 10 minutes, elapse between the time of the application of contrast medium Cb and the beginning of the third diagnostic recording Mb8. In particular less than 20 minutes, advantageously less than 17 minutes elapses between the time of the application of contrast medium Cb and the beginning of the third diagnostic recording Mb8.

This especially advantageously enables, in the third diagnostic recording Mb8, namely the delayed enhancement measurement, the late accumulation of the contrast medium in heart of the examination object to be examined. The fact that the application of contrast medium Cb takes place as early as possible in the second heart imaging, namely advantageously during the positioning of the examination object on the patient support facility of the magnetic resonance device, enables a waiting time between the application of contrast medium Cb and the delayed enhancement measurement to be advantageously shortened.

Advantageously, in the standardized execution sequence of the second heart imaging, almost all overview measurements and diagnostic measurements are made between the application of contrast medium Cb and the third diagnostic recording Mb8. This enables the third diagnostic recording Mb8 to be positioned as far away as possible in time from the application of contrast medium Cb, so that an especially suitable accumulation of the contrast medium in the heart of the examination object is present for the delayed enhancement measurement. The waiting time between the application of contrast medium Cb and the third diagnostic recording Mb8 can be exploited especially meaningfully by the suitable temporal arrangement of the overview measurements Mb1, Mb2, Mb3, Mb4, Mb6, of the first diagnostic measurement Mb5 and the second diagnostic measurement Mb7. In this way it can be insured that the maximum imaging duration for the second heart imaging can be adhered to.

In accordance with the description of the second heart imaging in FIG. 2, the fourth diagnostic recording Mb9, the dynamic heart recording along the short axis measurement slices, is arranged in time after the delayed enhancement measurement. This enables the fourth diagnostic recording Mb9 to be positioned as far away as possible in time from the application of contrast medium Cb in the second heart imaging. In this way, at the ninth point in time Tb9 an accumulation of contrast medium in the heart of the examination object can already be further reduced. In this way a disruptive influence of the contrast medium administered to the examination object on the fourth diagnostic measurement data acquired in the fourth diagnostic recording Mb9 can be advantageously reduced.

In accordance with the description of the second heart imaging in FIG. 2, the first diagnostic recording Mb5, the dynamic heart recording along the long axis measurement slices, is arranged as a temporally first diagnostic recording after the application of contrast medium Cb. Here a possible disruptive influence of the contrast medium administered to the examination object on the first diagnostic measurement data is taken into account, in order to be able to keep the first imaging duration as short as possible. Based on the first diagnostic measurement data, recording parameters for the further diagnostic measurements, in particular a positioning of the short axis measurement slices, will namely be set.

The application of contrast medium Cc for the third heart imaging is in particular not undertaken before the start of the third heart imaging, but at the beginning of the eighth measurement block Mc8. In this way, in the eighth measurement block Mc8, a spread of the contrast medium administered to the examination object can be dynamically examined in the stress perfusion measurement.

The twelfth point in time Tc12 is selected in this case such that at least 6 minutes, in particular at least 8 minutes, advantageously at least 10 minutes, elapse between the time of the application of contrast medium Cc and the beginning of the sixth diagnostic recording Mc12. In this way, in the delayed enhancement measurement the late accumulation of the contrast medium in the heart of the examination object can be examined especially advantageously.

Advantageously, in the standardized execution sequence of the third heart imaging, all remaining diagnostic measurements Mc9, Mc10, Mc11 as well as those of the perfusion measurements and the first diagnostic recording Mc5 are carried out between the application of contrast medium Cc and the sixth diagnostic recording Mb12. In this way the sixth diagnostic recording Mc12 can be positioned as far away as possible in time from the application of contrast medium Cc, so that an especially suitable accumulation of the contrast medium in the heart of the examination object is present for the delayed enhancement measurement. The waiting time between the application of contrast medium Cc and the sixth diagnostic recording Mc12 can be exploited especially meaningfully by the suitable temporal arrangement of the remaining diagnostic measurements Mc9, Mc10, Mc11. In this way it can be insured that the maximum imaging duration for the third heart imaging can be adhered to.

In accordance with the description of the third heart imaging in FIG. 3, the fifth diagnostic recording Mb11, the dynamic heart recording along the short axis measurement slices, is arranged in time directly before the delayed enhancement measurement. In this way the delayed enhancement measurement can be positioned further away in time from the application of contrast medium Cc and the imaging duration of the third heart imaging can be advantageously shortened. The fifth diagnostic recording Mb11 is still positioned as far away as possible in time from the application of contrast medium Cc in the third heart imaging, so that a disruptive influence of the contrast medium administered to the examination object on the fifth diagnostic measurement data acquired in the fifth diagnostic recording Mb11 is advantageously reduced as much as possible.

Relationship of Recording Parameters Between Diagnostic Recordings

The first diagnostic recording and the second diagnostic recording in particular have orientations along different heart axes. Thus only one recording of the first and second diagnostic recordings is carried out along the long axis and the other of the first and second diagnostic recordings along the short axis.

During the first diagnostic recording measurement slices in the heart of the examination object orthogonal to one another are acquired in particular. On the other hand, during the second diagnostic recording measurement slices in the heart of the examination object in parallel to one another are acquired in particular. The planning of an orientation of the measurement slices in parallel to one another, which are acquired during of the second diagnostic recording, can in this case be based especially advantageously on the acquisition of the measurement slices orthogonal to one another in the first diagnostic recording. Specifically for the first heart imaging, during the second diagnostic recording in particular, more than twice as many, preferably more than three times as many, measurement slices are acquired as are acquired during the first diagnostic recording.

The number of measurement slices acquired in the diagnostic recordings and the time resolution of the diagnostic measurement data is in particular selected so that the maximum imaging duration for the heart imaging is adhered to and at the same time an especially high diagnostic expressiveness is achieved. The user can be given an opportunity to modify the number of measurement slices and/or the time resolution of the diagnostic measurement data. Then in particular however such settings of the number of measurement slices and/or of the time resolution of the diagnostic measurement data, which lead to higher imaging durations than the predetermined maximum imaging duration are blocked for the user. If the number of user interactions is to be reduced, parameter settings, such as for example the number of measurement slices and/or the slice resolution and/or the pixel resolution and/or the time resolution, can also be predetermined.

The described harmonization of the recording parameters for the recordings along the long axis by comparison with the recording along the short axis makes it possible to speed up the execution sequence of the heart imaging such that the acquisition of the diagnostic measurement data needed for the assessment of the heart, for example the heart function, of the examination object is made possible within a maximum imaging duration. At the same time a high diagnostic image quality of the recorded diagnostic measurement data and/or a simple reproducibility of this image quality in a series of examinations in accordance with the heart imaging can be achieved.

Specifically in the second heart imaging all other diagnostic recordings Mb7, Mb8, Mb9 except for the first diagnostic recording Mb5, are made from the short axis measurement slices. In this way all other diagnostic recordings Mb7, Mb8, Mb9 are planned into the second heart imaging based on the first diagnostic measurement data acquired in the first diagnostic recording Mb8. In addition, in the other diagnostic recordings Mb7, Mb8, Mb9, diagnostic measurement data can also be recorded along long axis measurement slices, as is the case in the case of the third diagnostic recording Mb8 shown in FIG. 2 for example.

Specifically in the second heart imaging and the third heart imaging a number of diagnostic recordings are made from a stack consisting of short axis measurement slices. In such cases the stack of short axis measurement slices is smaller in each case for the T1 mapping measurement in the second heart imaging or third heart imaging than for the dynamic CINE recording in the same heart imaging. Also, for the perfusion measurement in the third heart imaging, the stack of short axis measurement slices is smaller than for the dynamic CINE recording in the third heart imaging.

Relationship of the Durations Between the Diagnostic Recordings and Overview Recordings

In all heart imagings, in the case shown, there are four measurement blocks with overview recordings before the beginning of the fifth measurement block, which totaled up, last more than twice as long as the fifth measurement block with the first diagnostic recording. The fifth measurement block in particular needs more time when compared to the fourth measurement block.

In all heart imagings, in the case shown, the third measurement block and the fourth measurement block together in particular last longer than the first measurement block combined with the second measurement block. The third measurement block and the fourth measurement block are those measurement blocks of which the overview measurement data serves to define the orientation or the recording region of the long axis measurement slices. The first measurement block and the second measurement block on the other hand are those measurement blocks, on the basis of the overview measurement data of which a positioning of the heart in the isocenter of the magnetic resonance device takes place. Thus the measurement blocks in which the overview measurement data related to the definition of the long axis is recorded, last longer than the measurement blocks in which overview measurement data, which is not embodied for defining the long axis is recorded. Of the first three measurement blocks, in which overview measurement data is recorded, the third measurement block lasts for about the same time as the first two measurement blocks. Thus the third measurement block lasts far longer than each of the first two measurement blocks.

Specifically for the first heart imaging, the seventh measurement block Ba7 with the second diagnostic recording Ma1, in which the measurement data is recorded along the short axis, in particular has a shorter duration than the fifth measurement block Ba5 with the first diagnostic recording Ma5, in which the measurement data is recorded along the long axis. In particular the seventh measurement block Ba7 in the first heart imaging lasts for less than 80 percent, preferably less than 70 percent, in particular less than 60 percent of the duration of the fifth measurement block Ba5. This is primarily attributable to the time outlay for the third evaluation step Ea3 and the third user interaction, which occur during the fifth measurement block Ba5. The pure measurement time for the second diagnostic recording Ma7 in the first heart imaging is longer than the pure measurement time for the first diagnostic recording Ma5.

In the first heart imaging the measurement blocks Ba1, Ba2, Ba3, Ba4, Ba6 with the overview recordings Ma1, Ma2, Ma3, Ma4, Ma6, totaled up, have a duration that amounts to five thirds of the totaled-up duration of the measurement blocks Ba5, Ba7 with the diagnostic recordings Ma5, Ma7. The start of the fifth measurement block Ba5 of the first diagnostic recording Ma5 lies in this case at precisely a half of the overall imaging duration of the first heart imaging. In the first heart imaging the evaluation of the first diagnostic measurement data and second diagnostic measurement data in the fifth evaluation step, which takes place after the end of the imaging duration of the first heart imaging, have a duration that amounts to around a third of the imaging duration.

In the heart imagings with application of contrast medium Cb, Cc, namely the second heart imaging and the third heart imaging, the measurement blocks with the overview recordings, totaled up, have a duration that is shorter than the totaled-up duration of the measurement blocks with the diagnostic recordings.

Compressed Sensing

An acceleration technique is used in particular for acquisition of the diagnostic measurement data, in particular of the dynamic CINE heart recordings. Acceleration techniques are typically used for other diagnostic measurements and the overview measurements. Different acceleration techniques known to the person skilled in the art, such as for example a parallel imaging, can be employed for acquisition of the diagnostic measurement data. In particular the use of a compressed sensing acceleration technique is conceivable. The compressed sensing acceleration technique, which is advantageously used for acquisition of the diagnostic measurement data, can be used in combination with the different magnetic resonance sequences, which lead to the different contrast behaviors. The compressed sensing acceleration technique here is known to the person skilled in the art, so that it will not be discussed in any greater detail here. For an especially advantageous reconstruction of the diagnostic measurement data acquired by way of the compressed sensing acceleration technique a movement-dependent regularization can be employed, as is described in US 2014/0126796 A1. In this respect reference is made to US 2014/0126796 A1, wherein its contents are herewith fully included in this application and is hereby incorporated herein by reference, in its entirety. An advantageous compressed sensing acceleration technique can use an incoherent sampling of k-space data and/or a partial Fourier technique. Here, as is described in US 2014/0086469 A1, for the reconstruction of the diagnostic measurement data, there is especially advantageously a use of weighted Haar Wavelets, in order to be able to exploit spatial and/or temporal correlations in the diagnostic measurement data. In this respect reference is made to US 2014/0086469 A1, wherein its contents are herewith fully included in this application and is hereby incorporated herein by reference, in its entirety.

The use of the compressed sensing acceleration technique can make it possible to record the diagnostic measurement data in an especially short recording time. By way of the compressed sensing acceleration technique a similar spatial and temporal resolution to conventional segmented recording techniques or real-time recording techniques can advantageously be achieved with a far shorter recording time. Precisely in the determination of a heart function, because of the high recording time usually needed, it can make particular sense to use the compressed sensing acceleration technique. The compressed sensing acceleration technique can in this way make it possible to acquire the diagnostic magnetic resonance measurement data in very few breathholds or in one breathing phase or when breathing freely. Thus an influence of the movement of the examination object on the diagnostic magnetic resonance measurement data can be greatly reduced. The use of the compressed sensing acceleration technique can also make possible a robust acquisition of the diagnostic magnetic resonance measurement data with uncooperative patients or patients who can only hold their breath for a short time or not at all or who have an irregular heartbeat or an arrhythmia.

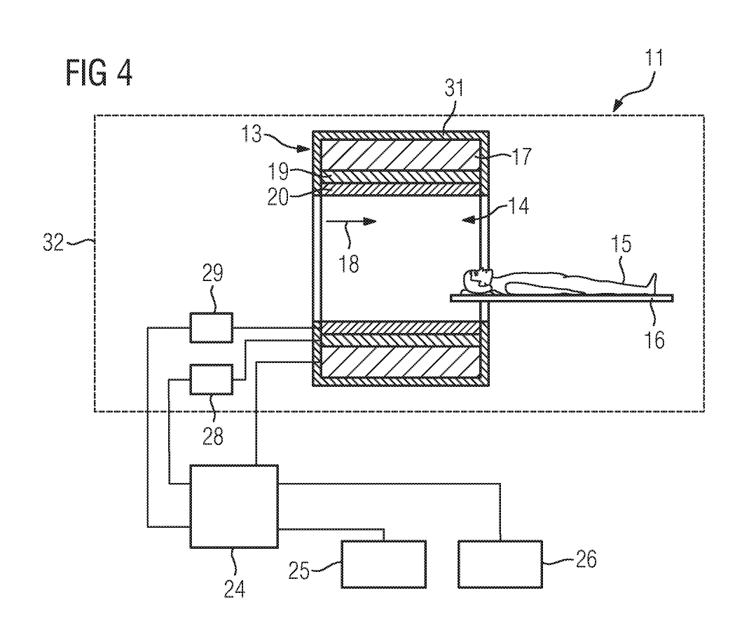

FIG. 4—Magnetic Resonance Device

FIG. 4 shows a schematic diagram of an inventive magnetic resonance device 11 for carrying out the heart imagings in accordance with FIG. 1-FIG. 3. The magnetic resonance device 11 comprises a detector unit formed by a magnet unit 13 with a main magnet 17 for creating a strong and in particular constant main magnetic field 18. In addition the magnetic resonance device 11 has a cylindrical patient receiving area 14 for a recording an examination object 15, in the present case a patient, wherein the patient receiving area 14 is surrounded cylindrically in a circumferential direction by the magnet unit 13. The patient 15 can be pushed via a patient support facility 16 of the magnetic resonance device 11 into the patient receiving area 14. For this purpose, the patient support facility 16 has a table, which is arranged movably inside the magnetic resonance device 11. The magnet unit 13 is screened from the outside by way of housing cladding 31 of the magnetic resonance device.

The magnet unit 13 also has a gradient coil unit 19 for creating magnetic field gradients, which is used for spatial encoding during imaging. The gradient coil unit 19 is activated via a gradient control unit 28. Furthermore the magnet unit 13 has a radio frequency antenna unit 20, which, in the case shown, is embodied as a body coil integrated permanently into the magnetic resonance device 11, and a radio frequency antenna control unit 29 for exciting a polarization, which occurs in the main magnetic field 18 created by the main magnet 17. The radio frequency antenna unit 20 is activated by the radio frequency antenna control unit 29 and irradiates radio frequency magnetic resonance sequences into an examination space, which is essentially formed by the patient receiving area 14. The radio frequency antenna unit 20 is further embodied for receiving magnetic resonance signals, in particular from the patient 15.

For controlling the main magnet 17, the gradient control unit 28 and the radio frequency antenna control unit 29, the magnetic resonance device 11 has a processing unit 24. The processing unit 24 controls the magnetic resonance device centrally, such as for example the carrying out of a predetermined imaging gradient echo sequence. Control information, such as for example imaging parameters, as well as reconstructed magnetic resonance images, can be provided on a display unit 25, of the magnetic resonance device 11 for a user. In addition the magnetic resonance device 11 has an input unit 26, by which information and/or parameters can be input by a user during a measurement process. The processing unit 24 can include the gradient control unit 28 and/or the radio frequency antenna control unit 29 and/or the display 25 and/or the input unit 26.

The magnetic resonance device 11 further comprises a measurement data acquisition unit 32. The measurement data acquisition unit 32 is formed in the present case by the magnet unit 13 together with the radio frequency antenna control unit 29 and the gradient control unit 28. The magnetic resonance device 11 is thus designed, together with the measurement data acquisition unit 32 and the processing unit 24, for carrying out an embodiment of an inventive method.

The magnetic resonance device 11 shown can of course comprise further components that magnetic resonance devices 11 usually have. A general way in which a magnetic resonance device 11 functions is also known to the person skilled in the art, so that a more detailed description of the further components will be dispensed with here.

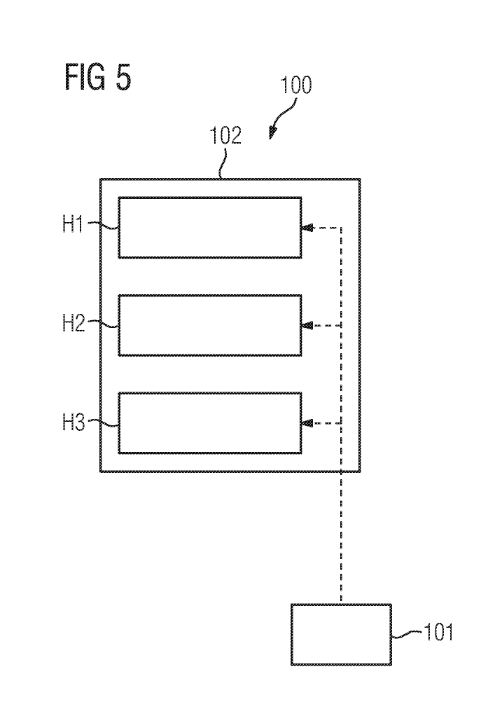

FIG. 5—Selection System

FIG. 5 shows a selection system 100, which makes it possible for a user to select a heart imaging to be carried out. The selection system 100 comprises a user interface, by which the user can select the heart imaging to be carried out. For this the user interface comprises a selection unit 101 and an output unit 102. The selection unit can in particular be embodied as the input unit 26 of the magnetic resonance device in accordance with FIG. 4. The output unit 102 can in particular be embodied as the display unit 25 of the magnetic resonance device 11 in accordance with FIG. 4. It is also conceivable, in specific cases, for the selection system 100 shown in FIG. 5 to be embodied separately from the magnetic resonance device 11.

The different heart imagings to be selected are displayed on the output unit 102, in particular together with or on a suitable control panel H1, H2, H3. In the case shown in FIG. 5, the first heart imaging, which is described in FIG. 1, is assigned to a first button H1 of the output unit 102, the second heart imaging, which is described in FIG. 2, is assigned to a second button H2 of the output unit 102 and the third heart imaging, which is described in FIG. 3, is assigned to a third button H1 of the output unit 102.

The presentation of the buttons H1, H2, H3 and the associated labeling can be embodied in accordance with a form appearing sensible to the person skilled in the art. The buttons H1, H2, H3 can be labeled for example with the diagnostic options of the respective heart imagings assigned to them. Thus for example the first button H1 can be labeled such that the associated first heart imaging is embodied for assessing a heart function of the examination object. The second button H2 can be labeled such that the associated second heart imaging is embodied for assessing a heart function and the possible presence of a non ischemic cardiomyopathy of the examination object. The third button H3 can be labeled such that the associated second heart imaging is embodied for assessing a heart function and the possible presence of an ischemic cardiomyopathy of the examination object. Furthermore the maximum imaging duration of the assigned heart imaging can be displayed for the buttons H1, H2, H3 in each case.

In this way the user can select a button H1, H2, H3 with the selection unit 101, in order to select the associated heart imaging to be carried out. In this way, the user, by actuating the first button H1, can select the first heart imaging for execution, by actuating the second button H2, can select the second heart imaging for execution and by actuating the third button H3, can select the third heart imaging for execution. The button can be selected via a procedure appearing sensible to the person skilled in the art, for example via a click, a double click, a Drag&Drop action, etc.

Of course other imaging execution sequences, possibly also of other areas of the body of the examination object, can be displayed on the output unit 102 and made available to the user for selection. The buttons H1, H2, H3 can even be arranged in a larger protocol tree, which comprises further imaging execution sequences to be selected.

After selection of a button H1, H2, H3 by the user via the selection unit 101, the associated heart imaging can be started. In this way information about selection of the button H1, H2, H3 by the selection system 100 can be transmitted to the magnetic resonance device 11. The selection of the button H1, H2, H3 can immediately initiate the start of the associated heart imaging. Advantageously however it will be made possible for the user first of all to enter patient-specific features for the respective heart imaging, before the imaging starts.

Of course it is conceivable for at least one additional diagnostic recording to be introduced into the heart imagings presented. This can lead to a lengthening of the imaging duration of the respective heart imagings. The possible additional at least one diagnostic recording can for example comprise a flow measurement and/or a coronary measurement.

Although the invention has been illustrated and described in greater detail by the preferred example embodiments, the invention is not however restricted by the disclosed examples and other variations can be derived herefrom by the person skilled in the art, without departing from the scope of protection of the invention.

1. A method for recording diagnostic measurement data of a heart of an examination object in a heart imaging, via a magnetic resonance device, comprising:
carrying out of a number of overview recordings of the heart of the examination object, wherein overview measurement data is acquired in the number of overview recordings; and
carrying out of a number of diagnostic recordings of the heart of the examination object based on the overview measurement data acquired, wherein diagnostic measurement data is acquired in the number of diagnostic recordings,
wherein at least two overview recordings, of the number of overview recordings, and at least two diagnostic recordings, of the number of diagnostic recordings, are carried out in a temporal execution sequence at least partly nested in one another, and
wherein a temporally first diagnostic recording, of the at least two diagnostic recordings, and a temporally second diagnostic recording, of the at least two diagnostic recordings, are carried out along respectively different heart axes of the examination object.

2. (canceled)

3. The method of claim 1, wherein, in the heart imaging, there are more than twice as many overview recordings before the temporally first diagnostic recording of the at least two diagnostic recordings as there are overview recordings between the temporally first diagnostic recording of the at least two diagnostic recordings and the temporally second diagnostic recording of the at least two diagnostic recordings.

4. The method of claim 1, wherein the number of overview recordings amounts to a maximum of six.

5. (canceled)

6. The method of claim 1, wherein measurement slices in the heart of the examination object, orthogonal to one another, are acquired in the temporally first diagnostic recording of the at least two diagnostic recordings and measurement slices in the heart of the examination object, in parallel to one another, are acquired in the temporally second diagnostic recording of the at least two diagnostic recordings.

7. The method of claim 6, wherein a planning of the measurement slices in parallel to one another, is based on the measurement slices orthogonal to one another, acquired in the temporally first diagnostic recording.

8. The method of claim 1, wherein a number of measurement blocks exist with overview recordings before the a beginning of a measurement block with the temporally first diagnostic recording of the number of diagnostic recordings, wherein the number of measurement blocks with the overview recordings, totaled up, last more than twice as long as the measurement block with the temporally first diagnostic recording.

9. The of claim 1, wherein, at a beginning of the heart imaging, there is at least one overview measurement for positioning the heart in an isocenter of the magnetic resonance device and at least one overview measurement for defining at least one of an orientation and a recording region of long axis measurement slices.

10. The method of claim 9, wherein at least one measurement block with the at least one overview measurement for defining at least one of the orientation and of the recording region of the long axis measurement slices last for a relatively longer time than the at least one measurement block with the at least one overview measurement for positioning the heart in the isocenter of the magnetic resonance device.

11. The method of claim 1, wherein the carrying out of at least one part of the number of diagnostic recordings comprises a use of a compressed sensing acceleration technique.

12. The method of claim 1, wherein there are a maximum of five user interactions during the heart imaging.

13. The method of claim 1, wherein a combined figure for the number of overview recordings and the number of diagnostic recordings is at least twice as large as a figure for a number of user actions occurring during the heart imaging.

14. The method of claim 1, wherein there is precisely one user interaction between the temporally first diagnostic recording of the number of diagnostic recordings and the temporally second diagnostic recording of the number of diagnostic recordings.

15. The method of claim 1, wherein there are at least twice as many user interactions before a beginning of the temporally first diagnostic recording of the number of diagnostic recordings as there are user interactions between the temporally first diagnostic recording and the temporally second diagnostic recording of the number of diagnostic recordings.

16. The method of claim 1, wherein there are more automatic evaluation steps than user interactions during the heart imaging.

17. The method of claim 1, wherein the user is automatically presented with suggestions for a necessary user interaction, simply be acceptable or modifiable by the user for the user interaction.

18. The method of claim 1, wherein, for a necessary user interaction, the user is automatically provided on a display unit with instructions for at least one of the user interaction and with suitable tools for the user interaction.

19. The method of claim 1, wherein a maximum imaging duration threshold for the heart imaging is stored, wherein parameters are only settable by a user for the heart imaging such that the maximum imaging duration threshold will not be exceeded with the imaging parameters set.

20. The method of claim 1, wherein the heart imaging is a first heart imaging and the number of diagnostic recordings exclusively comprise the following diagnostic recordings:
a first diagnostic recording, embodied as a dynamic heart recording along relatively long axis measurement slices of the heart, and
a second diagnostic recording, embodied as a dynamic heart recording along relatively short axis measurement slices of the heart.

21. The method of claim 20, wherein a first maximum imaging duration threshold, amounting to a maximum of 12 minutes, is stored for the first heart imaging.

22. The method of claim 21, wherein the first maximum imaging duration amounts to a maximum of 6 minutes.

23. The method of claim 20, wherein, in the first heart imaging, the second diagnostic recording follows on in time from the first diagnostic recording.

24. The method of claim 23, wherein, in the first heart imaging, the relatively short axis measurement slices are planned based on the diagnostic measurement data acquired in the first diagnostic recording.

25. The method of claim 20, wherein, in the first heart imaging, more than twice as many relatively short axis measurement slices are acquired in the second diagnostic recording as there are relatively long axis measurement slices acquired in the first diagnostic recording.

26. The method of claim 20, wherein, in the first heart imaging, a figure for the number of overview recordings is at least twice as great as a figure for the number of diagnostic recordings.

27. The method of claim 20, wherein the first heart imaging is carried out without application of contrast medium.

28. The method of claim 20, wherein, in the first heart imaging, the measurement block with the second diagnostic recording has a relatively shorter duration than a measurement block with the first diagnostic recording.

29. The method of claim 20, wherein, in the first heart imaging, measurement blocks with the overview recordings, totaled up, need a relatively longer duration than measurement blocks with the diagnostic recordings totaled up.

30. The method of claim 20, wherein a start of a measurement block with the first diagnostic recording occurs at a half of the imaging duration of the first heart imaging.

31. The method of claim 20, wherein, in the first heart imaging, an evaluation of first diagnostic measurement data and second diagnostic measurement data after an end of an imaging duration of the first heart imaging has a duration that amounts to more than a quarter of the imaging duration.

32. The method of claim 20, wherein a compressed sensing acceleration technique is used in the first heart imaging for the first diagnostic recording and the second diagnostic recording.

33. The method of claim 20, wherein the diagnostic measurement data recorded in the first heart imaging is embodied for assessing a heart function of the examination object.

34. A method for recording diagnostic measurement data of a heart of an examination object in a heart imaging via a magnetic resonance device, comprising:
carrying out of a number of overview recordings of the heart of the examination object, wherein overview measurement data is acquired in the number of overview recordings; and
carrying out of a number of diagnostic recordings of the heart of the examination object based on the overview measurement data acquired, wherein diagnostic measurement data is acquired in the number of diagnostic recordings, wherein the heart imaging is a second heart imaging and the number of diagnostic recordings exclusively comprise:
a first diagnostic recording, embodied as a dynamic heart recording along a relatively long axis measurement slices of the heart,
a second diagnostic recording, embodied as a T1-mapping measurement,
a third diagnostic recording, embodied as a delayed enhancement measurement, and
a fourth diagnostic recording, embodied as a dynamic heart recording along a relatively short axis measurement slices of the heart, and
wherein the second diagnostic recording and the third diagnostic recording are made in a time between the first diagnostic recording and the fourth diagnostic recording in the second heart imaging.

35. The method of claim 34, wherein a second maximum imaging duration, amounting to a maximum of 18 minutes, is stored for the second heart imaging.

36. The method of claim 35, wherein the second maximum imaging duration amounts to a maximum of 10 minutes.

37. (canceled)

38. The method of claim 34, wherein there is an application of contrast medium before a start of a first measurement block in the second heart imaging.

39. The method of claim 38, wherein at least 10 minutes elapse between a time of the application of contrast medium and a beginning of the third diagnostic recording in the second heart imaging.

40. The method of claim 34, wherein the first diagnostic recording and the second diagnostic recording are carried out in the time before the third diagnostic recording and the fourth diagnostic recording is carried out in the time after the third diagnostic recording in the second heart imaging.

41. The method of claim 40, wherein the fourth diagnostic recording is placed in the second heart imaging such that a contrast medium accumulation in the heart of the examination object is already reduced again by a time of the fourth diagnostic recording.

42. The method of claim 34, wherein measurement blocks with the overview recordings, totaled up, have a duration that is relatively shorter than a totaled-up duration of measurement blocks with the diagnostic recordings in the second heart imaging.

43. The method of claim 34, wherein the diagnostic measurement data recorded in the second heart imaging is embodied for assessing a heart function and a possible presence of a non-ischemic cardiomyopathy in the examination object.

44. A method for recording diagnostic measurement data of a heart of an examination object in a heart imaging via a magnetic resonance device, comprising:
carrying out of a number of overview recordings of the heart of the examination object, wherein overview measurement data is acquired in the number of overview recordings; and
carrying out of a number of diagnostic recordings of the heart of the examination object based on the overview measurement data acquired, wherein diagnostic measurement data is acquired in the number of diagnostic recordings, wherein the heart imaging is a third heart imaging and the number of diagnostic recordings exclusively comprise:
a first diagnostic recording, embodied as a dynamic heart recording along a relatively long axis measurement slices of the heart,
a second diagnostic recording, embodied as a perfusion measurement,
a fourth diagnostic recording, embodied as a T1 mapping measurement,
a fifth diagnostic recording, embodied as a dynamic heart recording along a relatively short axis measurement slices of the heart, and
a sixth diagnostic recording, embodied as a delayed enhancement measurement,
wherein the fourth diagnostic recording and the fifth diagnostic recording are made in a time between the second diagnostic recording and the sixth diagnostic recording in the third heart imaging.

45. The method of claim 44, wherein a second maximum imaging duration, amounting to a maximum of 22 minutes, is stored for the third heart imaging.

46. The method of claim 45, wherein the third maximum imaging duration amounts to a maximum of 15 minutes.

47. The method of claim 44, wherein there is an application of contrast medium in a time after the first diagnostic recording and in a time before the second diagnostic recording in the third heart imaging.

48. The method of claim 47, wherein at least 6 minutes elapse between a time of the application of contrast medium and a beginning of the sixth diagnostic recording in the third heart imaging.

49. (canceled)

50. The method of claim 49, wherein a third diagnostic recording, embodied as a thorax recording in at least one of coronal and transversal measurement slices, is made additionally in a time between the second diagnostic recording and the sixth diagnostic recording.

51. The method of claim 44, wherein measurement blocks with the overview recordings, totaled up, have a duration that is relatively shorter than a totaled-up duration of measurement blocks with the diagnostic recordings in the third heart imaging.

52. The method of claim 44, wherein the diagnostic measurement data recorded in the third heart imaging is embodied for assessing a heart function, a possible presence of a non-ischemic cardiomyopathy and the a possible presence of an ischemic cardiomyopathy of the examination object.

53. A magnetic resonance device, comprising,
a measurement data acquisition unit; and
a processing unit, wherein the magnetic resonance device is designed to record diagnostic measurement data of a heart of an examination object in a heart imaging via at least one of the measurement data acquisition unit and processing unit performing at least:
carrying out of a number of overview recordings of the heart of the examination object, wherein overview measurement data is acquired in the number of overview recordings; and
carrying out of a number of diagnostic recordings of the heart of the examination object based on the overview measurement data acquired, wherein diagnostic measurement data is acquired in the number of diagnostic recordings, wherein the heart imaging is a third heart imaging and the number of diagnostic recordings exclusively comprise:
a first diagnostic recording, embodied as a dynamic heart recording along a relatively long axis measurement slices of the heart,
a second diagnostic recording, embodied as a perfusion measurement,
a fourth diagnostic recording, embodied as a T1 mapping measurement,
a fifth diagnostic recording, embodied as a dynamic heart recording along a relatively short axis measurement slices of the heart, and
a sixth diagnostic recording, embodied as a delayed enhancement measurement,
wherein the fourth diagnostic recording and the fifth diagnostic recording are made in a time between the second diagnostic recording and the sixth diagnostic recording in the third heart imaging.

54. A non-transitory computer readable medium storing a computer program, directly loadable into a memory of a programmable processing unit of a magnetic resonance device, including program code segments for carrying out the method of claim 1 when the computer program is executed in the processing unit of the magnetic resonance device.

55. A non-transitory computer readable medium storing a computer program, directly loadable into a memory of a programmable processing unit of a magnetic resonance device, including program code segments for carrying out the method of claim 34 when the computer program is executed in the processing unit of the magnetic resonance device.

56. A non-transitory computer readable medium storing a computer program, directly loadable into a memory of a programmable processing unit of a magnetic resonance device, including program code segments for carrying out the method of claim 44 when the computer program is executed in the processing unit of the magnetic resonance device.

57. The method of claim 3, wherein a number of overview recordings amounts to a maximum of six.
